(19) United States
(12) Patent Application Publication
(10) Pub. No.: US 2007/0248628 A1
(43) Pub. Date: Oct. 25, 2007

(54) Immunogens in cancer stem cells

(76) Inventors: Lorraine Holowach Keller, Pipersville, PA (US); Ramila Philip, Ivyland, PA (US)

Correspondence Address: Lorraine H. Keller;Immunotope Inc., The Pennsylvania Biotechnology Center, 3805 Old Easton Road, Doylestown, PA, 18902, US

(21) Appl. No.: 11/633,858

(22) Filed: Dec. 5, 2006

Related U.S. Application Data
(62) Division of application No. 60/742,586, filed on Dec. 6, 2005.

(51) Int. Cl. A61K 39/00; A61P 35/00; C07H 21/02; C07K 2/00; C12Q 1/00

(52) U.S. Cl. 424/277.1; 435/4; 530/300; 536/23.1

(57) ABSTRACT

The present invention relates to compositions and methods for the prevention, treatment, and diagnosis of cancer, especially cancer stem cells. The invention discloses peptides, polypeptides, and polynucleotides that can be used to stimulate a CTL response against cancer cells, especially cancer stem cells.

### CROSS-REFERENCE TO RELATED APPLICATIONS

This application claims priority of U.S. Provisional Application No. 60/742,586 filed Dec. 6, 2005.

### FIELD OF THE INVENTION

The present invention relates generally to the field of immunogens whose structures incorporate polypeptides comprising epitopic peptides derived from proteins expressed by cancer cells, preferably cancer stem cells, and to uses of said immunogens for the treatment, prevention and diagnosis of cancer, most preferably ovarian and breast cancer.

There is no cure for cancer. Standard treatments include surgery, chemotherapy and radiation therapy, which are only temporarily effective in slowing the progression of advanced stage disease. Seventy to ninety percent of cancer patients die from recurrence of disease because standard treatments fail to eradicate residual tumor cells. These cells eventually metastasize, killing the patient. The only way to cure cancer is to eliminate all residual tumor cells throughout the body to prevent recurrence. Ideally, treatment would selectively kill tumor cells and would not adversely affect normal, healthy cells. One means to selectively kill tumor cells is by active immunotherapy, which activates the patient's own immune system to recognize and kill tumor cells.

It has long been recognized that only a very small percentage of cells in many types of tumors are capable of immortal growth (Southam C and Brunschwig, A. Cancer 14:971-78 (1960); Reya T., et al. Nature 414:105-111 (2001)). There is growing evidence that cancer stem cells are present in a number of cancers, including leukemia (Dick J. E. Cancer Cells 3:39-48 (1991)), glioma (Singh S. et al. Nature 432:396-401(2004)), colon (Reya T., and H. Clevers Nature 434:843-850 (2005)), ovarian (Bapat S., et al. Cancer Res. 65:3025-3029 (2005)), breast (Al-Hajj M., et al. Proc. Nat. Acad. Sci. 100:3983-3988 (2003)) lung (Kim, C., et al., Cell 121:823-836 (2005)), prostate (Schalken J. and van Leenders G. Curr. Opinion Genetics and Development 14:48-54 (2004)), retinoblastoma (Dyer, M. and R. Brenner Nature Rev. Cancer 5:91-101 (2005)) and hepatocellular carcinoma (Hsia et al. Hepatology 16:1327-33 (1992)). The cancer stem cell hypothesis (Reya T. et al. Nature 414:105-111 (2002)) teaches that certain tumors originate from and persist due to mutations in stem cells that undergo neoplastic transformation, resulting in unregulated, immortal proliferation>Transformed, immortal stem cells are referred to as cancer stem cells (CSC). CSC typically constitute only a small subset of the total tumor cell population, for example composing only approximately 1/1000 to 1/5000 cells in lung tumors and 1/1,000,000 in leukemia cells (Marx J. Science 301:1308-1310 (2003)). In breast tumors, distinct classes of cells have been isolated, including CSC clonal lines that continuously proliferate, and cell populations that terminally differentiate after only a few divisions (Al-Hajj et al. Proc. Nat Acad. Sci. 100:3983-3988 (2003)). Cancer stem cells have been isolated from established tumor cell lines ( Gudjonsson, T., R. Villadsen, H. L. Nielsen, L. Ronnov-Jessen, M. J. Bissell, and O. W. Petersen. Genes Dev 16:693 (2002); Kondo, T., T. Setoguchi, and T. Taga. Proc Natl Acad Sci USA 101:781 (2004); Ponti, D., A. Costa, N. Zaffaroni, G. Pratesi, G. Petrangolini, D. Coradini, S. Pilotti, M. A. Pierotti, and M. G. Daidone. Cancer Res 65:5506 (2005)) and retain the same phenotype as the tumors from which they were originally isolated.

Evidence from several tumor types suggests that pathways prominent in normal stem cell function, notably Wnt, Notch, and Ssh, become ‘dysregulated’ in CSC. Therefore understanding the neoplastic changes that occur in these pathways should lead to an understanding of how tumors form, how they are maintained, how they evade standard treatments, and how they can be eradicated. There is a need in the cancer field to develop therapeutic strategies to target and kill immortal cancer cells, preferably CSC, and to prevent their recurrence, in order to cure patients of the disease.

Characterizing the properties of tumors, and in particular immortal cancer stem cells, which are important therapeutic targets, has proven extremely difficult. Differential gene expression has been widely used to identify cancer-associated gene activity to identify cancer-associated gene products having potential therapeutic or diagnostic value. However, this approach has generated ‘unmanageably large amounts of data’ (Rhodes, D. et al, Proc. Nat. Acad. Sci. 101:9309-9314 (2004)). Typically, large gene expression profile databases are generated from normal and cancerous tissues to identify genes that are differentially expressed in cancer; that is, -up- or down-regulated in tumor cells compared to normal cells. In order to understand the fundamental underlying mechanisms of cancer, considerable effort has also been invested in identifying gene products common to multiple tumor types (Rhodes et al. ibid.). However, the vast amount of data that have been generated by microarray analysis has magnified the amount of information available without solving the critical problem of determining which essential features of neoplastic transformation and progression are characteristic of CSC, and which properties of CSC can be exploited to develop targeted and potent therapeutics to eradicate CSC and to prevent tumor recurrence and metastasis. Because tumors are highly heterogeneous, and cancer stem cells are only present in very small quantities, gene expression studies over-represent the cell populations that terminally differentiate and mask the characteristics of the very small proportion of CSC in the tumor that continually proliferate. This invention addresses the need for identifying candidates for therapeutics that can target CSC and destroy the small fraction of neoplastic cancer cells that are responsible for recurrence and metastasis.

Existing targeted drug treatment strategies, for example small molecules and antibodies, target gene products of differentiated pathways, which shrink tumors, but are not effective in killing CSC. This invention provides methods and compositions for targeting and destroying CSC to eradicate tumors and prevent recurrence and metastasis.

Immunotherapy can be used to efficiently target and kill tumor cells through the therapeutic application of tumor rejection antigens, which can be expressed as peptides, oligopeptides or polypeptides. Immunotherapy can be used post-surgery and chemotherapy to eliminate residual cancer cells throughout the body. Alternatively, immunotherapy can also be used at early stages of tumor development, Stage I/II, to induce tumor rejection and eliminate the tumor cells from the body while the tumor burden is low. To achieve a clinically effective outcome for the patient, it is essential to treat with antigens that will stimulate an immune response against immortal cells, including cancer stem cells.

The mammalian immune system has evolved a variety of mechanisms to protect the host from diseased cells. An important component of this response is mediated by cells referred to as cytotoxic T lymphocytes (CTLs), which are specialized immune cells that function primarily by recognizing and killing diseased cells, and also by secreting soluble molecules referred to as cytokines that can mediate a variety of effects on the immune system.

Evidence suggests that immunotherapy designed to stimulate a CTL response specifically against cancer cells will be effective in controlling and eliminating cancer cells (Clark, C and Vonderheide R. Clin. Cancer Res. 11:5333-5336 (2005)). The largest number of reports of human tumor-reactive CTLs have concerned cancers (Rosenberg et al., Nature Medicine 10:909-915 (2004); Hwu P. and Friedman R., J. Immunotherapy 25:189-201 (2002); Boon, T. et al., Ann. Rev. Immunol., 12:337-365, (1994)). The ability of tumor-specific CTLs to mediate tumor regression, in both human (Rosenberg, S. A. et al., N. Engl. J. Med., 319:1676-1680, (1988)) and animal models (Celluzzi, C. M. et al., J. Exp. Med., 183:283-287, (1996); Mayordomo, J. I. et al., Nat. Med., 1:1297-1302, (1995); Zitvogel, L. et al., J. Exp. Med., 183:87-97, (1996)), suggests that methods directed to increasing CTL activity would likely have a beneficial effect with respect to recognition and elimination of tumor cells.

In order for CTLs to kill diseased cells or to secrete cytokines in response to a tumor cell, the CTL must first recognize that cell as being cancerous. This process involves the interaction of the T cell receptor, located on the surface of the CTL, with what is generically referred to as a Major Histocompatibility Complex (MHC)-peptide complex that is located on the surface of the tumor cell. MHC-encoded molecules have been subdivided into two types, and are referred to as class I and class II MHC-encoded molecules. Background information on cellular immunity and its use in immunotherapy is found in Janeway, C. et al. (Immunobiology: The Immune System in Health and Disease, Chapter 8. T Cell-Mediated Immunity. 5^{th} Edition. Chapter ISBN 0 8185-3642-x (2001)).

In the human immune system, MHC molecules are referred to as human leukocyte antigens (HLA). Within the MHC, located on chromosome six, are three different genetic loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, and HLA-B8 are examples of different class I MHC molecules that can be expressed from these loci. The present disclosure involves peptides that are associated with the HLA-A1, HLA-A2, or HLA-A11 molecules, HLA-A1 supertypes, HLA-A2 supertypes, and HLA-A11 supertypes and with the gene and protein that gives rise to these peptides. A supertype is a group of HLA molecules that present at least one shared epitope.

The peptides that associate with the MHC molecules can either be derived from proteins made within the cell, in which case they typically associate with class I MHC molecules (Rock, K. L. and Golde, U., Ann. Rev. Immunol., 17:739-779, (1999)) or they can be derived from proteins that are acquired from outside of the cell, in which case they typically associate with class II MHC molecules (Watts, C., Ann. Rev. Immunol., 15:821 850, (1997)). Peptides that evoke a cancer-specific CTL response most typically associate with class I MHC molecules; however certain cancer antigen-related peptides that elicit a CTL response may be associated with class II MHC molecules, for example, Her2/neu. The peptides that associate with a class I MHC molecule are typically nine amino acids in length, but can vary from a minimum length of eight amino acids to a maximum of fourteen amino acids in length. A class I MHC molecule with its bound peptide, or a class II MHC molecule with its bound peptide, is referred to as a MHC-peptide complex.

The process by which intact proteins or polypeptides are degraded into peptides and oligopeptides is referred to as antigen processing. Two major pathways of antigen processing occur within cells (Shastri N. et al. Ann. Rev. Immunol. 20:463-93 (2002); Rock, K. L. and Golde, U., Ann. Rev. Immunol., 17:739-779, (1999); Watts, C., Ann. Rev. Immunol., 15:821-850, (1997)). One pathway, which is largely restricted to cells that are antigen presenting cells such as dendritic cells, macrophages, and B cells, degrades proteins that are typically phagocytosed or endocytosed into the cell. Peptides derived in this pathway typically bind to class II MHC molecules. A second pathway of antigen processing is present in essentially all cells of the body. This second pathway primarily degrades proteins that are made within the cells, and the peptides derived from this pathway primarily bind to class I MHC molecules. Antigen processing by this latter pathway involves polypeptide synthesis and proteolysis in the cytoplasm. The peptides produced are then transported into the endoplasmic reticulum of the cell, associate with newly synthesized class I MHC molecules, and the resulting MHC-peptide complexes are then transported to the cell surface. Peptides derived from membrane and secreted proteins have also been identified. In some cases these peptides correspond to the signal sequence of the proteins that are cleaved from the protein by the signal peptidase. In other cases, it is thought that some fraction of the membrane and secreted proteins are transported from the endoplasmic reticulum into the cytoplasm where processing subsequently occurs.

Once bound to the class I MHC molecule and displayed on the surface of a cell, the peptides are recognized by antigen-specific receptors on CTLs. Mere expression of the class I MHC molecule itself is insufficient to trigger the CTL to kill the target cell: the antigenic peptide must be bound to the class I MHC molecule. Several methods have been developed to identify MHC-associated peptides recognized by CTL, each method relying on the ability of a CTL to recognize and kill only those cells expressing the appropriate class I MHC molecule with the peptide bound to it (Rosenberg, S. A., Immunity, 10:281-287, (1999)). Such peptides can be derived from a non-self source, such as a pathogen (for example, following the infection of a cell by a bacterium or a virus) or from a self-derived protein within a cell, such as a cancerous cell. Examples of sources of self-derived proteins in cancerous cells have been reviewed (Shastri N. et al. Ann. Rev. Immunol. 20:463-93 (2002), Gilboa, E., Immunity, 11:263-270, (1999); Rosenberg, S. A., Immunity, 10:281-287, (1999)) and include: (i) mutated genes; (ii) aberrantly expressed genes such as an alternative open reading frame or through an intron-exon boundary; (iii) genes that are selectively expressed in only the tumor; and (iv) normal differentiation genes that are expressed in the tumor and the normal cellular counterpart. The same proteins are processed by individuals of all HLA types; however the proteins are cleaved at different sites to generate different MHC-associated peptide epitopes.

Four different methodologies have typically been used for identifying the peptides that are recognized by CTLs. These are: (i) the genetic method; (ii) motif analysis; (iii) SErological analysis of REcombinant cDNA expression libraries (SEREX); and (iv) direct extraction and identification of native, endogenously processed and presented MHC-associated peptide epitopes. The genetic method is an approach in which progressively smaller subsets of cDNA libraries from tumor cells are transfected into cells that express the appropriate MHC molecule but not the tumor-specific epitope. The molecular clones encoding T cell epitopes are identified by their ability to reconstitute tumor specific T cell recognition of transfected cells. The exact T cell epitope is then identified by a combination of molecular subcloning and the use of synthetic peptides based on the predicted amino acid sequence. Such methods, however, are susceptible to inadvertent identification of cross-reacting peptides, and are not capable of identifying important post-translational modifications.

Motif analysis involves scanning a protein for peptides containing known class I MHC binding motifs, followed by synthesis and assay of the predicted peptides for their ability to be recognized by tumor-specific CTL. This approach requires prior knowledge of the protein from which the peptides are derived. This approach is also greatly hampered by the fact that not all of the predicted peptide epitopes are presented on the surface of a cell (Yewdell, J. W. and Bennink, J. R., Ann. Rev. Immunol., 17:51-88, (1999)), thus additional experimentation is required to determine which of the predicted epitopes is useful.

The SEREX approach relies on using antibodies in the serum of cancer patients to screen cDNA expression libraries for a clone that expresses a protein recognized by the antibody. This methodology presumes that an antibody response will necessarily have developed in the presence of a T cell response, and thus, the identified clone is a candidate that may encode a protein that can be recognized by T cells.

Direct immunoprecipitation involves a combination of cellular immunology and mass spectrometry, and uses native autoantigens to capture autoantibodies in cancer patient serum. This approach involves the actual identification of CTL epitopes by sequencing the naturally occurring peptides associated with class I MHC molecules extracted directly from tumor cells. In this approach, cells are first lysed in a detergent solution, the peptides associated with the class I MHC molecules are purified, and the peptides fractionated by high performance liquid chromatography (HPLC). The peptides are then used to reconstitute recognition by tumor-specific CTLs on a non-tumor cell expressing the appropriate MHC molecules. Sequencing is readily performed by tandem mass spectrometry (Henderson, R. A. et al., Proc. Natl. Acad. Sci. U.S.A, 90:10275-10279, (1993); Hogan, K. T. et al., Cancer Res., 58:5144-5150, (1998); Hunt, D. F. et al., Science, 255:1261-1263, (1992); Slingluff, C. L. Jr et al., J. Immunol., 150:2955-2963, (1993)).

Immunization with cancer-derived, class I MHC-encoded molecule-associated peptides, or with a precursor polypeptide or protein that contains the peptide, or with a gene that encodes a polypeptide or protein containing the peptide, are forms of immunotherapy that can be employed in the treatment of cancer. These forms of immunotherapy require that immunogens be identified so that they can be formulated into an appropriate vaccine. Although a variety of cancer-derived antigens have been identified (Rosenberg, S. A., Immunity, 10:281-287, (1999)), not all of these are appropriate for broad-based immunotherapy as the expression of some peptides is limited to the tumor derived from a specific patient. Furthermore, the number of class I MHC molecules from which tumor-derived peptides have been discovered is largely restricted to HLA-A2. Thus, it would be useful to identify additional peptides that complex with class I MHC molecules other than HLA-A2. Such peptides would be particularly useful in the treatment of cancer patients who do not express the HLA-A2 molecule, HLA-A1 or HLA-A11 antigens, HLA-A1 supertypes, HLA-A2 supertypes and HLA-A11 supertypes, for example. It is also particularly useful to identify antigenic peptides that are derived from different original proteins, even if the derived peptides associate with the same class I MHC molecule. Because an active immune response can result in the outgrowth of tumor cells that have lost the expression of a particular precursor protein for a given antigenic peptide, it is advantageous to stimulate an immune response against peptides derived from more than one protein, as the chances of the tumor cell losing the expression of more than one protein is the multiple of the chances of losing each of the individual proteins.

It is critical that the immunization process result in effective recognition of cancer cells by CTL. The only means by which CTL can distinguish cancerous cells is through recognition of tumor-associated antigens, also called tumor rejection antigens, presented on the tumor cell surface as peptides associated with MHC molecules. In the body, tumor cells may exist and proliferate undetected by the immune system, a phenomenon known as tolerance. Effective immunotherapeutic vaccines ‘break tolerance’ and activate CTL by providing at least one, and preferably more than one tumor-associated antigens present on the tumor cell surface, most preferably on the cancer stem cell surface, to Antigen Presenting Cells (APC). Examples of APC include dendritic cells (DC) and macrophages. APC provide exposure of the CTL to the antigen and a costimulatory signal that activates naive T cells to become CTL that recognize and destroy tumor cells that display the tumor-associated antigen in association with MHC molecules on their surfaces. Because CTL have memory, they can recognize and kill tumor cells over long periods of time, thus preventing recurrence and metastasis. However, it is essential that immunotherapeutic vaccination activates CTL to the same tumor-associated antigens presented on the surfaces of the tumor cells, most preferably cancer stem cells, in the context of MHC molecules. An explanation of tolerance and mechanisms for breaking tolerance are found in Janeway et al.

Embryonic and somatic stem cells and stem cell progenitors are capable of expressing MHC Class I and Class II molecules (Drukker M. et al., Proc Nat. Acad. Sci. 99:9864-9869 (2002); Harris P. et al., Blood 87:5104-5112 (1996); Smit et al., Proc. Nat. Acad. Sci. 95:10152-10157 (1998); Al Nimer, F. Neuroreport 15:1871-5 (2004)). Smit et al. observed T cells specifically directed against CD34+ Chronic Myeloid Leukemia (CML) progenitors and a subsequent antileukemic effect. Odeberg et al. (J. Neuroimmunol. 161:1-11 (2005)) reported high MHC Class I and Class II expression but low immunogenicity in neural precursors expanded in vitro. In certain stem cells, expression of MHC antigens can be stimulated, for example with interferon-gamma, a cytokine that upregulates MHC expression (Hori J., et al. Stem Cells 21:405-416 (2003)).

MHC expression in stem cells has been studied in the context of regenerative medicine, transplantation, graft immunogenicity and cell-based therapy. These studies do not consider or suggest a role for MHC Class I or Class II antigen expression in the context of characterization of cancer stem cells, or the identification of potential targets for immunotherapy against cancer stem cells, or for immunotherapeutic treatment for the treatment or prevention of cancer recurrence or metastasis. The present invention describes methods and compositions for cancer immunotherapy specifically intended to identify immunogens, that is, peptides, oligopeptides and their parent or source proteins or polypeptides that stimulate CTL recognition for the selective destruction of cancer cells, most preferably destruction of cancer stem cells. The present invention also contemplates identifying immunogens on terminally differentiated cancer cells that arise from cancer stem cells, as such immunogens can be used to kill differentiated cells and to shrink the tumor.

Immunotherapy for the purposes of this invention includes immunotherapeutic vaccines and antibody therapeutics. The invention also contemplates the use of certain antigens as cancer diagnostics and drug targets.

Immunization with cancer-derived, class I MHC-encoded molecule-associated peptides, or with a precursor polypeptide or protein that contains the peptide, or with a gene that encodes a polypeptide or protein containing the peptide, are forms of immunotherapy that can be employed in the treatment of cancer. These forms of immunotherapy require that immunogens be identified so that they can be appropriately formulated for administration to a cancer patient.

It is also particularly useful to identify antigenic peptides that are derived from a variety of different parent proteins with different functions, most preferably proteins involved in tumor survival, tumor growth and tumor metastasis. Because an active immune response can result in the outgrowth of tumor cells that have lost the expression of a particular precursor protein for a given antigenic peptide, it is advantageous to stimulate an immune response against multiple different antigens from more than one parent protein, as the chances of the tumor cell losing the expression of more than one protein is the multiple of the chances of losing each of the individual proteins. Even if an antigenic peptide is identified only in association with a single type of HLA molecule, the information is valuable for immunotherapy and immunization because as long as the source protein is known to undergo antigen processing, that source protein or polypeptide or fragments of the protein can be administered to patients having multiple HLA types, wherein each patient's Antigen Presenting Cells will properly process the protein in association with the patient's own HLA type to elicit a CTL response.

### SUMMARY OF THE INVENTION

The present invention relates to a process for identifying immunogens that elicit an immune response against cancer cells, more specifically against cancer stem cells. These immunogens can be used to treat patients who already have the disease, or prophylactically to prevent disease.

The present invention relates to Immunogens comprising polypeptides with amino acid sequences comprising epitopic sequences selected from the sequences of SEQ ID NO: 1-125 and 126-242 and which immunogens facilitate a cytotoxic T lymphocyte (CTL)-mediated immune response against cancers, most preferably cancers consisting of cancer stem cells. The present invention also relates to nucleic acid molecules that encode for the polypeptides and/or the full length proteins from which the polypeptides are derived, of such immunogens, and which can also be used to facilitate an immune response against cancer and cancer stem cells.

The present invention also relates to isolated oligopeptides or peptides comprising at least one epitopic peptide comprising the amino acid sequence of SEQ ID NO: 1-125, said oligopeptide or peptide consisting of 8 to about 50 amino acid residues, wherein said oligopeptide or peptide binds to class I MHC molecules or can be processed to bind to class I MHC molecules.

The present invention also relates to an isolated polypeptide of SEQ ID NO: 126 to 242, wherein said polypeptide binds to class I MHC molecules or can be processed to bind to class I MHC molecules.

The present invention provides compositions comprising the immunogen described herein, and polynucleotides that direct the synthesis of such polypeptides, whereby the oligopeptides and polypeptides of such immunogens are capable of inducing a CTL response against cells expressing a protein comprising an epitopic sequence of at least one of SEQ ID NO: 1-125 and 126-242. The cells are usually cancer cells, preferably ovarian or breast carcinomas and most preferably ovarian or breast carcinoma cancer stem cells expressing such proteins.

The present invention further relates to polynucleotides comprising the gene coding for a polypeptide of the SEQ ID NO: 125-242 disclosed herein. This invention relates to methods for identifying cancer stem cell immunogens that elicit a CTL response comprising obtaining tumorigenic cancer stem cells, identifying MHC-associated peptides that are present on said tumorigenic cancer stem cells, formulating a pharmaceutical preparation of one or more of immunogens comprising proteins or polypeptides with sequences selected from the group SEQ ID NO: 1-242 that bind to class I MHC molecules or can be processed to bind to class I MHC molecules, and administering said pharmaceutical preparation to a cancer patient to elicit a tumor-rejection response.

The invention further relates to methods for identifying cancer stem cell antigens that elicit a CTL response comprising obtaining cancer stem cells, identifying MHC-associated peptides that are present in said tumorigenic cancer stem cells, identifying the source protein or expression product from which said peptides were derived selected from the group consisting of SEQ ID NO: 126-242, formulating a pharmaceutical preparation of one or more of said source proteins or expression products, and administering said pharmaceutical preparation to a cancer patient to elicit a tumor-rejection response.

The invention further relates to pharmaceutical preparations wherein said preparations comprise polynucleotides coding for proteins, peptides or polypeptides with sequences selected from the group SEQ ID NO: 1-242 that bind to class I MHC molecules or can be processed to bind to class I MHC molecules that are capable of activating a tumor rejection CTL response in vitro or in vivo in a cancer patient.

The invention further relates to compositions for the treatment of cancer comprising CTL-reactive antigens isolated from cancer stem cells consisting of at least one tumor-rejection antigen that bind to class I MHC molecules or can be processed to bind to class I MHC molecules that stimulates a CTL response against tumor cells.

The invention further relates to methods and compositions for diagnosis of cancer comprising immunogens selected from the group consisting of SEQ ID NO: 126 to 242.

The invention further relates to methods and compositions for developing antibody therapeutics for treatment of cancer characterized by the presence of cancer stem cells comprising identifying CTL-reactive antigens for at one MHC-associated tumor-rejection antigen that binds to class I MHC molecules or can be processed to bind to class I MHC molecules present in said cancer stem cell.

The present invention also provides methods that comprise contacting a lymphocyte, especially a CTL, with an immunogen of the invention under conditions that induce a CTL response against a tumor cell, and more specifically against a cancer cell and most specifically against a cancer stem cell. The methods may involve contacting the CTL with the immunogenic peptide in vivo, in which case the peptides, polypeptides, and polynucleotides of the invention are used as vaccines, and will be delivered as a pharmaceutical composition comprising a pharmaceutically acceptable carrier and the immunogen, typically along with an adjuvant or one or more cytokines.

Alternatively, the immunogens of the present invention can be used to induce a CTL response in vitro. The generated CTL can then be introduced into a patient with cancer, said cancer characterized by the presence of cancer stem cells, more specifically cancer, colorectal carcinoma, ovarian carcinoma, breast carcinoma, lung carcinoma, or prostate carcinoma. Alternatively, the ability to generate CTL in vitro could serve as a diagnostic for cancer generally, including ovarian carcinoma, breast carcinoma, colorectal carcinoma, lung carcinoma, or prostate carcinoma.

The present invention also relates to a process for identifying immunogens that can be used to develop antibody therapeutics for cancer, most preferably therapeutics directed to destroying cancer stem cells.

The present invention also relates to a process for identifying immunogens that can be used as diagnostics and prognostics for cancer, preferably for the detection and monitoring of cancer stem cells, including autoantibody-based diagnostics for detection of autoantigens that characterize the cancer cells as cancer stem cells.

### Definitions

As used herein and except as noted otherwise, all terms are defined as given below.

The term “peptide” is used herein to designate a series of amino acid residues, connected one to the other typically by peptide bonds between the alpha-amino and carbonyl groups of the adjacent amino acids. The peptides are typically 9 amino acids in length, but can be as short as 8 amino acids in length, and as long as 14 amino acids in length.

The term “oligopeptide” is used herein to designate a series of amino acid residues, connected one to the other typically by peptide bonds between the alpha-amino and carbonyl groups of the adjacent amino acids. The length of the oligopeptide is not critical to the invention as long as the correct epitope or epitopes are maintained therein. The oligopeptides are typically less than about 50 amino acid residues in length, and greater than about 14 amino acids in length.

The term “polypeptide” designates a series of amino acid residues, connected one to the other typically by peptide bonds between the alpha-amino and carbonyl groups of the adjacent amino acids. The length of the polypeptide is not critical to the invention as long as the correct epitopes are maintained. In contrast to the terms peptide or oligopeptide, the term polypeptide is meant to refer to protein molecules of longer than about 50 residues in length.

A peptide, oligopeptide, protein, or polynucleotide coding for such a molecule is “imnunogenic” (and thus an “immunogen” within the present invention) if it is capable of inducing an immune response. In the case of the present invention, immunogenicity is defined in one way as the ability to induce a CTL-mediated response. Thus, an “immunogen” would be a molecule that is capable of inducing an immune response, and in the case of the present invention, a molecule capable of inducing a CTL response. An immunogen is a molecule that binds to class I MHC molecules or can be processed to bind to class I MHC molecules.

A “T cell epitope” or “epitopic peptide” is a peptide or oligopeptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the epitope molecule are not buried in the structure of the class II MHC molecule peptide-binding cleft as they are in the class I MHC molecule peptide-binding cleft.

There are three different genetic loci that encode for class I MHC molecules: HLA-A, HLA-B, and HLA-C. HLA-A1, HLA-A2, and HLA-A11 are examples of different class I MHC molecules that can be expressed from these loci. The present invention also involves peptides that are associated with HLA-A1 supertypes, HLA-A2 supertypes, and HLA-A11 supertypes. A supertype is a group of HLA molecules that present at least one shared epitope. MHC molecule peptides that have been found to bind to one member of the MHC allele supertype family (A1 for example) are thought to be likely to bind to other members of the same supertype family (A32 for example; see Table 1, below.

TABLE 1
HLA Supertypes

| Supertype | Motif | Genotypes |
|---|---|---|
| A1 | x[TI(SVLM)] | A*0101, A*0102, A*2501, A*2601, |
| | xxxxxx[WFY] | A*2604, A*3201, A*3601, A*4301, |
| | | A*8001 |
| A2 | x[LIVMATQ] | A*0201, A*0202, A*0203, A*0204, |
| | xxxxxx[LIVMAT] | A*0205, A*0206, A*0207, A*6802, |
| | | A*6901 |
| A3 | x[AILMVST] | A*0301, A*1101, A*3101, A*3301, |
| | xxxxxx[RK] | A*6801 |
| A24 | x[YF(WIVLMT)] | A*2301, A*2402, A*2403, A*2404, |
| | xxxxxx[FI(YWLM)] | A*3001, A*3002, A*3003 |
| B7 | x[P]xxxxxx | B*0702, B*0703, B*0704, B*0705, |
| | [ALIMVFWY] | B*1508, B*3501, B*3502, B*3503, |
| | | B*51, B*5301, B*5401, B*5501, |
| | | B*5502, B*5601, B*5602, B*6701, |
| | | B*7801 |
| B27 | x[RKH]xxxxxx | B*1401, B*1402, B*1503, B*1509, |
| | [FLY(WMI)] | B*1510, B*1518, B*2701, B*2702, |
| | | B*2703, B*2704, B*2705, B*2706, |
| | | B*2707, B*2708, B*3801, B*3802, |
| | | B*3901, B*3902, B*3903, B*3904, |
| | | B*4801, B*4802, B*7301 |
| B44 | x[E(D)]xxxxxx | B*18, B*3701, B*4001, B*4006, |
| | [FWYLIMVA] | B*4101, B*4402, B*4403, B*4501, |
| | | B*4901, B*5001 |
| B58 | x[AST]xxxxxx | B*1516, B*1517, B*5701, B*5702, |
| | [FWY(LIV)] | B*58 |
| B62 | x[QL(IVMP)] | B*1301, B*1302, B*1501, B*1502, |
| | xxxxxx[FWY(MIV)] | B*1506, B*1512, B*1513, B*1514, |
| | | B*1519, B*1521, B*4601, B*52 |

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

The term “nucleotide sequence” refers to a heteropolymer of deoxyribonucleotides. The nucleotide sequence encoding for a particular peptide, oligopeptide, or polypeptide may be naturally occurring or may be synthetically constructed. Generally, DNA segments encoding the peptides, polypeptides, and proteins of this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “source protein” means that polypeptide that is the natural translation product of its gene and any nucleic acid sequence coding equivalents resulting from genetic code degeneracy and thus coding for the same amino acid(s). A ‘parent peptide’ or ‘parent protein’ as used herein designates a series of amino acid residues that comprise or naturally occur within the source protein.

The term “expression product” means that polypeptide or source protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “DNA segment” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, by using a cloning vector. Such segments are provided in the form of an open reading frame uninterrupted by internal nontranslated sequences, or introns, which are typically present in eukaryotic genes. Sequences of non-translated DNA may be present downstream from the open reading frame, where the same do not interfere with manipulation or expression of the coding regions.

The term “primer” means a short nucleic acid sequence that is paired with one strand of DNA and provides a free 3′OH end at which a DNA polymerase starts synthesis of a deoxyribonucleotide chain.

The term “promoter” means a region of DNA involved in binding of RNA polymerase to initiate transcription.

The term “open reading frame (ORF)” means a series of triplets coding for amino acids without any termination codons and is a sequence (potentially) translatable into protein.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polynucleotides, and recombinant or immunogenic polypeptides, disclosed in accordance with the present invention may also be in “purified” form. The term “purified” does not require absolute purity; rather, it is intended as a relative definition, and can include preparations that are highly purified or preparations that are only partially purified, as those terms are understood by those of skill in the relevant art. For example, individual clones isolated from a cDNA library have been conventionally purified to electrophoretic homogeneity. Purification of starting material or natural material to at least one order of magnitude, preferably two or three orders, and more preferably four or five orders of magnitude is expressly contemplated. Furthermore, the claimed polypeptide which has a purity of preferably 0.001%, or at least 0.01% or 0.1%; and even desirably 1% by weight or greater is expressly contemplated.

The nucleic acids and polypeptide expression products disclosed according to the present invention, as well as expression vectors containing such nucleic acids and/or such polypeptides, may be in “enriched form.” As used herein, the term “enriched” means that the concentration of the material is at least about 2, 5, 10, 100, or 1000 times its natural concentration (for example), advantageously 0.01%, by weight, preferably at least about 0.1% by weight. Enriched preparations of about 0.5%, 1%, 5%, 10%, and 20% by weight are also contemplated. The sequences, constructs, vectors, clones, and other materials comprising the present invention can advantageously be in enriched or isolated form.

The term “active fragment” means a fragment that generates an immune response (i.e., has immunogenic activity) when administered, alone or optionally with a suitable adjuvant, to an animal, such as a mammal, for example, a human, a rabbit or a mouse, such immune response taking the form of stimulating a CTL response within the recipient animal, such as a human. Alternatively, the “active fragment” may also be used to induce a CTL response in vitro.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. This means that any such fragment will necessarily contain as part of its amino acid sequence a segment, fragment or portion, that is substantially identical, if not exactly identical, to a sequence of SEQ ID NO: 1 to 125, which correspond to the naturally occurring, original or “parent” proteins of the SEQ ID NO: 126-242. When used in relation to polynucleotides, such terms refer to the products produced by treatment of said polynucleotides with any of the common endonucleases.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

The term “autoantibody” is used herein to designate an antibody that reacts with the cells, tissues, or native proteins of the host in which it is produced.

The term “autoantigen” is used herein to designate an immunogen that stimulates the production of autoantibodies in the host in which it is produced.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated Percent Identity is less than the specified Percent Identity.

### DETAILED DESCRIPTION OF THE INVENTION

The present invention relates generally to immunogens and immunogenic compositions, and methods of use therefore, for the prevention, treatment, and diagnosis of cancer, more specifically cancers harboring cancer stem cells, including ovarian, breast, colorectal, lung, prostate and liver carcinomas. Disclosed according to the invention are immunogens comprising proteins or polypeptides with sequences selected from the group SEQ ID NO: 1 to 242 whose amino acid sequences comprise one or more epitopic oligopeptides or peptides that bind to MHC molecules or can be processed to bind to MHC molecules. In addition, the invention further relates to DNA or RNA polynucleotides coding for peptides or oligopeptides selected from the group SEQ ID NO: 1-242, and that bind to MHC molecules or can be processed to bind to MHC molecules to stimulate a CTL response against cancer, and more specifically cancers containing cancer stem cells, especially ovarian and breast cancers.

In accordance with the present invention, there are disclosed specific oligopeptide sequences with amino acid sequences representing epitopic peptides and immunogenic oligopeptide sequences of at least about 8 amino acids in length, preferably about 9 amino acids in length, up to about 14 amino acids in length and may be present as part of a larger structure, such as an oligopeptide, polypeptide or source protein.

The polypeptides forming the immunogens of the present invention may consist of amino acid sequences comprising at least one and possibly two, three, four, or more sequences of about 8 to 11 residues in length. Said polypeptides can have any manner of amino acid substitutions and can be of any desired length so long as they have immunogenic activity in that they are able, under a set of conditions known to those in the art, to elicit in vitro or in vivo activation of Cytotoxic T lymphocytes (CTL), (i.e., a CTL response) against a presentation of a cancer specific protein, especially a cancer stem cell-associated protein, especially when such proteins are presented in association with MHC molecules, such as where said proteins are presented in vitro or in vivo by an antigen presenting cell (APC). The amino acid stretches may differ in amino acid sequence by at least one amino acid residue, for example a conservative amino acid residue or amino acids of the same general chemical character, such as where they are hydrophobic amino acids. The proteins and polypeptides forming the immunogens of the present invention can be naturally occurring or may be synthesized chemically.

The present invention is also directed to an isolated polypeptide, especially one having immunogenic activity, the sequence of which comprises within it one or more stretches comprising any two or more of the sequences of an MHC-associated peptide or oligopeptide and in any relative quantities and wherein said sequences may differ by one or more amino acids so long as the peptide stimulates a CTL response, in any given stretch of 8 to about 11 amino acid residues. In other embodiments, combinations and permutations of epitopic sequences may be part of an immunogen of the present invention or of such a polypeptide so long as any such polypeptide stimulates a CTL response.

Peptides, oligopeptides or polypeptides as disclosed herein may be prepared by methods well known to those skilled in the art. (Grant, G. A., Synthetic Peptides: A User's Guide, 1992, W. H. Freeman and Company, New York; Coligan, J. E. et al, Current Protocols in Protein Science, 1999, John Wiley & 5 Sons, Inc., New York).

Besides the peptide sequences identified as associated with MHC molecules, the proteins and polypeptides forming the immunogens of the present invention may also comprise one or more other immunogenic amino acid stretches known to be associated with cancer, and more specifically with carcinomas, including ovarian, carcinoma, colorectal carcinoma, breast carcinoma, lung carcinoma, hepatocellular or prostate carcinoma, and most specifically with cancer stem cells associated with carcinomas, including ovarian carcinoma, colorectal carcinoma, breast carcinoma, lung carcinoma, hepatocellular or prostate carcinoma. These immunogens may stimulate a CTL response in individuals of any HLA type.

The immunogens of the present invention can be in the form of a composition of one or more of the different immunogens and wherein each immunogen is present in any desired relative abundance. Such compositions can be homogeneous or heterogeneous with respect to the individual immunogenic peptide components present therein, having only one or more than one of such peptides.

The oligopeptides and polypeptides useful in practicing the present invention may be derived by fractionation of naturally occurring proteins by methods such as protease treatment, or they may be produced by recombinant or synthetic methodologies that are well known and clear to the skilled artisan (Ausubel, F. M. et al, Current Protocols in Molecular Biology, 1999, John Wiley & Sons, Inc., New York; Coligan, J. E. et al, Current Protocols in Protein Science, 1999, John Wiley & Sons, Inc., New York; Molecular Cloning: A Laboratory Manual, 1989, Cold Spring Harbor Laboratory Press, Cold Spring Harbor). The polypeptide may comprise a recombinant or synthetic polypeptide in which peptide or oligopeptide sequences may also be present in multiple copies.

Thus, oligopeptides and polypeptides of the present invention may have one, two, three, or more such immunogenic peptides within the amino acid sequence of said oligopeptides and polypeptides, and said immunogenic peptides, or epitopes, may be the same or may be different, or may have any number of such sequences wherein some of them are identical to each other in amino acid sequence while others within the same polypeptide sequence are different from each other and said epitopic sequences may occur in any order within said immunogenic polypeptide sequence. The location of such sequences within the sequence of a polypeptide forming an immunogen of the invention may affect relative immunogenic activity.

In addition, immunogens of the present invention may comprise more than one polypeptide comprising the amino acid sequences disclosed herein. Such polypeptides may be part of a single composition or may themselves be covalently or non-covalently linked to each other.

The immunogenic peptides disclosed herein may also be linked directly to, or through a spacer or linker to: an immunogenic carrier such as serum albumin, tetanus toxoid, keyhole limpet hemocyanin, dextran, or a recombinant virus particle; an immunogenic peptide known to stimulate a T helper cell type immune response; a cytokine such as interferon gamma or GMCSF; a targeting agent such as an antibody or receptor ligand; a stabilizing agent such as a lipid; or a conjugate of a plurality of epitopes to a branched lysine core structure, such as the so-called “multiple antigenic peptide” described in (Posnett, D. N. et al., J. Biol. Chem., 263:1719-1725, (1988)); a compound such as polyethylene glycol to increase the half life of the peptide; or additional amino acids such as a leader or secretory sequence, or a sequence employed for the purification of the mature sequence. Spacers and linkers are typically comprise relatively small, neutral molecules, such as amino acids and which are substantially uncharged under physiological conditions. Such spacers are typically selected from the group of nonpolar or neutral polar amino acids, such as glycine, alanine, serine and other similar amino acids. Such optional spacers or linkers need not comprise the same residues and thus may be either homo- or hetero-oligomers. When present, such linkers will commonly be of length at least one or two, commonly three, four, five, six, and possibly as much as or even up to 20 residues (in the case of amino acids). In addition, such linkers need not be composed of amino acids but any oligomeric structures will do as well so long as they provide the correct spacing so as to optimize the desired level of immunogenic activity of the immunogens of the present invention. The immunogen may therefore take any form that is capable of eliciting a CTL response. In addition, the immunogenic peptides of the present invention may be part of an immunogenic structure via attachments other than conventional peptide bonds. Thus, any manner of attaching the peptides of the invention to an immunogen of the invention, such as an immunogenic polypeptide as disclosed herein, could provide an immunogenic structure as claimed herein.

Thus, immunogens, such as polypeptides of the invention, are structures that contain the peptides disclosed according to the present invention but such immunogenic peptides may not necessarily be attached thereto by the conventional means of using ordinary peptide bonds. The immunogens of the present invention simply contain such peptides as part of their makeup, but how such peptides are to be combined to form the final immunogen or combination of immunogens is left to the talent and expertise of the user and is in no way restricted or limited by the disclosure contained herein.

The peptides that are naturally processed and bound to a class I MHC molecule, and which are recognized by a tumor-specific CTL, need not be the optimal peptides for stimulating a CTL response. See, for example, (Parkhurst, 25 M. R. et al., J. Immunol., 157:2539-2548, (1996); Rosenberg, S. A. et al., Nat. Med., 4:321-327, (1998)). Thus, there can be utility in modifying a peptide, such that it more readily induces a CTL response. Generally, peptides may be modified at two types of positions. The peptides may be modified at amino acid residues that are predicted to interact with the class I MHC molecule, in which case the goal is to create a peptide that has a higher affinity for the class I MHC molecule than does the parent peptide. The peptides can also be modified at amino acid residues that are predicted to interact with the T cell receptor on the CTL, in which case the goal is to create a peptide that has a higher affinity for the T cell receptor than does the parent peptide. Both of these types of modifications can result in a variant peptide that is related to a parent peptide, but which is better able to induce a CTL response than is the parent peptide. As used herein, the term “parent peptide” means a peptide or oligopeptide sequence identified as being associated with a MHC molecule.

The parent peptides disclosed herein can be modified by the substitution of one or more residues at different, possibly selective, sites within the peptide chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.” Conservative substitutions are herein defined as exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gin); Group 3-polar, positively charged residues (His, Arg, Lys); Group 4-large, aliphatic, nonpolar residues (Met, Leu, lie, Val, Cys); and Group 4-large, aromatic residues (Pine, Tyr, Trp).

Less conservative substitutions might involve the replacement of one amino acid by another that has similar characteristics but is somewhat different in size, such as replacement of an alanine by an isoleucine residue. Highly nonconservative replacements might involve substituting an acidic amino acid for one that is polar, or even for one that is basic in character. Such radical substitutions cannot, however, be dismissed as potentially ineffective since chemical effects are not totally predictable and radical substitutions might well give rise to serendipitous effects not otherwise predictable from simple chemical principles. Of course, such substitutions may involve structures other than the common L-amino acids. Thus, D-amino acids might be substituted for the L-amino acids commonly found in the antigenic peptides of the invention and yet still be encompassed by the disclosure herein. In addition, amino acids possessing non-standard R groups (i.e., R groups other than those found in the common 20 amino acids of natural proteins) may also be used for substitution purposes to produce immunogens and immunogenic polypeptides according to the present invention.

If substitutions at more than one position are found to result in a peptide with substantially equivalent or greater immunogenic activity as defined below, then combinations of those substitutions can readily be tested using well-known methods to determine whether the combined substitutions result in additive or syngeneic effects on the immunogenicity of the peptide.

Based on cytotoxicity assays, an epitope is considered substantially identical to the reference peptide if it has at least 10% of the antigenic activity of the reference peptide as defined by the ability of the substituted peptide to reconstitute the epitope recognized by a CTL in comparison to the reference peptide. Thus, when comparing the lytic activity in the linear portion of the effector: target curves with equimolar concentrations of the reference and substituted peptides, the observed percent specific killing of the target cells incubated with the substituted peptide should be equal to that of the reference peptide at an effector:target ratio that is no greater than 10-fold above the reference peptide effector:target ratio at which the comparison is being made.

Preferably, when the CTL-specific peptides are tested against the substituted peptides, the peptide concentration at which the substituted peptides achieve half the maximal increase in lysis relative to background is no more than about 1 millimolar, preferably no more than about 1 micromolar, more preferably no more than about 1 nanomolar, and still more preferably no more than about 100 picomolar, and most preferably no more than about 10 picomolar. It is also preferred that the substituted peptide be recognized by CTLs from more than one individual, at least two, and more preferably three individuals.

Thus, the epitopes of the present invention may be identical to naturally occurring tumor-associated or tumor-specific peptide epitopes or may include epitopes that differ from the reference peptide, as long as they have substantially identical immunogenic activity and can bind to MHC Class I molecules or be processed to bind to MHC molecules.

The immunogenic peptides and polypeptides of the invention can be prepared synthetically, by recombinant DNA technology, or they can be isolated from natural sources such as tumor cells expressing the parent protein product.

The polypeptides and oligopeptides disclosed herein can be synthesized in solution or on a solid support in accordance with conventional techniques. Various automated peptide synthesizers are commercially available and can be used in accordance with known protocols. See, for example, (Grant, G. A., Synthetic Peptides: A User's Guide, 1992, W. H. Freeman and Company, New York; Coligan, J. E. et al, Current Protocols in Protein Science, 1999, John Wiley 5 & Sons, Inc., New York). Fragments of polypeptides of the invention can also be synthesized as intermediates in the synthesis of a larger polypeptide.

Recombinant DNA technology may be employed wherein a nucleotide sequence that encodes an immunogenic peptide or polypeptide of interest is inserted into an expression vector, transformed or transfected into an appropriate host cell, and cultivated under conditions suitable for expression. These procedures are well known in the art to the skilled artisan, as described in (Coligan, J. E. et al, Current Protocols in Immunology, 1999, John Wiley & Sons, Inc., New York; Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Thus, recombinantly produced peptides or polypeptides can be used as the immunogens of the invention.

The coding sequences for peptides of the length contemplated herein can be synthesized on commercially available automated DNA synthesizers using protocols that are well known in the art. See for example, (Grant, G. A., Synthetic Peptides: A User's Guide, 1992, W. H. Freeman and Company, New York; Coligan, J. E. et al, Current Protocols in Protein Science, 1999, John Wiley & Sons, Inc., New York). The coding sequences can also be modified such that a peptide or polypeptide will be produced that incorporates a desired amino acid substitution. The coding sequence can be provided with appropriate linkers, be ligated into suitable expression vectors that are commonly available in the art, and the resulting DNA or RNA molecule can be transformed or transfected into suitable hosts to produce the desired fusion protein. A number of such vectors and suitable host systems are available, and their selection is left to the skilled artisan. For expression of the fusion proteins, the coding sequence will be provided with operably linked start and stop codons, promoter and terminator regions, and a replication system to provide an expression vector for expression in the desired host cell. For example, promoter sequences compatible with bacterial hosts are provided in plasmids containing convenient restriction sites for insertion of the desired coding sequence. The resulting expression vectors are transformed into suitable bacterial hosts. Of course, yeast, insect, and mammalian host cells may also be used, employing suitable vectors and control sequences. Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell.

Introduction of the construct into the host cell can be effected by calcium phosphate transfection, DEAE-Dextran mediated transfection, or electroporation (Ausubel, F. M. et al, Current Protocols in Molecular Biology, 1999, John Wiley & Sons, Inc., New York; Molecular Cloning: A Laboratory Manual, 1989, Cold Spring Harbor Laboratory Press, Cold Spring Harbor). Such cells can routinely be utilized for assaying CTL activity by having said genetically engineered, or recombinant, host cells express the immunogenic peptides of the present invention. Various mammalian cell culture systems can also be employed to express recombinant protein. Examples of mammalian expression systems include the COS-7 lines of monkey kidney fibroblasts, described by Giuzman, Cell, 23:175 (1981), and other cell lines capable of expressing a compatible vector, for example, the HEK293, NIH3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking non-transcribed sequences. DNA sequences derived from the SV40 splice, and polyadenylation sites may be used to provide the required nontranscribed genetic elements.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography.

Protein refolding steps can be used, as necessary, in completing configuration of the mature protein. High performance liquid chromatography (HPLC) can be employed for final purification steps.

Compositions suitable for stimulating an immune response in vivo include immunotherapeutic vaccines with one or more peptides, oligonucleotides or polypeptides of the invention, or with polynucleotides coding for one or more peptides, oligonucleotides or polypeptides of the invention, can be accomplished using any of a variety of antigen delivery systems well known in the art that direct the peptides, oligonucleotides or polypeptides to the site of lymphoid cells where the selected immunogens are directly exposed to antigen presenting cells in order to elicit a CTL response against the vaccine components in vivo in the patient. Examples of delivery vehicles include liposomes, bacterial and viral vectors and heat shock proteins. Adjuvants include but are not limited to GM-CSF (Granulocyte Monocyte Colony Stimulating Factor), Listeria, LAG3 and heat shock proteins. Recombinant or synthetic peptides of the invention may be mixed with an adjuvant and directly injected into the patient. Thus, one or more peptides of the invention can be provided to antigen presenting cells in such a fashion that the peptides associate with class I MHC molecules and are presented on the surface of the antigen presenting cell, and consequently are available to stimulate a CTL response, against cancer cells, and preferably against cancer stem cells.

The immunogenic peptides of the present invention may be used to elicit CTLs ex vivo from either healthy individuals or from cancer patients with cancers such as ovarian carcinoma, breast carcinoma, colorectal carcinoma, lung carcinoma, or prostate carcinoma. Such responses are induced by incubating in tissue culture the individual's CTL precursor lymphocytes together with a source of antigen presenting cells and the appropriate immunogenic peptide. Examples of suitable antigen presenting cells include dendritic cells, macrophages, and activated B cells. Typically, the peptide at concentrations between 10 and 40 ug/ml would be pre-incubated with the antigen presenting cells for periods ranging from 1 to 18 hours. Beta-2-microglobulin (4 g/ml) can be added during this time period to enhance binding. The antigen presenting cells may also be held at room temperature during the incubation period (Ljunggren, H.-G. et al., Nature, 346:476-480, (1990)) or pretreated with acid (Zeta, H. J., Lil et al., Hum. Immunol., 39:79-86, (1994)) to promote the generation of denatured class I MHC molecules which can then bind the peptide. The precursor CTLs (responders) are then added to the antigen presenting cells to which the immunogenic peptide has bound (stimulators) at responder to stimulator ratios of between 5:1 and 50:1, and most typically between 10:1 and 20:1. The co cultivation of the cells is carried out at 37° C. in RPMI 1640, 10% fetal bovine serum, 2 mM L-glutamine, and IL-2 (5-20 Units/ml). Other cytokines, such as IL-1, IL-7, and IL-12 may also be added to the culture.

Fresh IL-2-containing media is added to the cultures every 2-4 days, typically by removing one-half the old media and replenishing it with an equal volume of fresh media. After 7-10 days, and every 7-10 days thereafter, the CTL are re-stimulated with antigen presenting cells to which immunogenic peptide has been bound as described above. Fresh IL-2-containing media is added to the cells throughout their culture as described above. Three to four rounds of stimulation, and sometimes as many five to eight rounds of stimulation, are required to generate a CTL response that can then be measured in vitro. The above-described protocol is illustrative only and should not be considered limiting.

Many in vitro CTL stimulation protocols have been described and the choice of which one to use is well within the knowledge of the skilled artisan. The peptide-specific CTL can be further expanded to large numbers by treatment with anti-CD3 antibody. For example, see (Riddell, S. R. and Greenberg, P. D., J. Immunol. Methods, 128:189-201, (1990); Walter, E. A. et al., N. Engl. J. Med., 333:1038-1044, (1995)).

Antigen presenting cells that are to be used to stimulate a CTL response are typically incubated with peptide of an optimal length, most commonly a nonapeptide, that allows for direct binding of the peptide to the class I MHC molecule without additional processing. Larger oligopeptides and polypeptides are generally ineffective in binding to class I MHC molecules as they are not efficiently processed into an appropriately sized peptide in the extracellular milieu. However, a variety of approaches are known in the art that allow oligopeptides and polypeptides to be exogenously acquired by a cell, which then allows for their subsequent processing and presentation by a class I MHC molecule.

Representative, but non-limiting examples of such approaches include electroporation of the molecules into the cell (Harding, C. H. III, Eur. J. Immunol., 22:1865-1869, (1992)), encapsulation of the molecules in liposomes which are fused to the cells of interest (Ready, R. et al., J. Immunol. Methods, 141:157-163, (1991)), or osmotic shock, in which the molecules are taken up via pinocytosis (Moore, M. W. et al., Cell, 54:777-785, (1988)). Thus, oligopeptides and polypeptides that comprise one or more of the peptides of the invention can be provided to antigen presenting cells in such a fashion that they are delivered to the cytoplasm of the cell, and are subsequently processed to allow presentation of the peptides by MHC molecules.

Antigen presenting cells suitable for stimulating an in vitro CTL response that is specific for one or more of the peptides of the invention can also be prepared by introducing polynucleotide vectors encoding the sequences into the cells. These polynucleotides can be designed such that they express only a single peptide of the invention, multiple peptides of the invention, or even a plurality of peptides of the invention. A variety of approaches are known in the art that allow polynucleotides to be introduced and expressed in a cell, thus providing one or more peptides, oligopeptides or polypeptides of the invention to the class I MHC molecule binding pathway. Representative, but non-limiting examples of such approaches include the introduction of plasmid DNA through particle-mediated gene transfer or electroporation (Tuting, T. et al., J. Immunol., 160:1139-1147, (1998)), or the transduction of cells with an adenovirus expressing the polynucleotide of interest (Perez-Diez, A. et al., Cancer Res., 58:5305-5309, (1998)). Thus, oligonucleotides that code for one or more of the peptides, oligopeptides or polypeptides of the invention can be provided to antigen presenting cells in such a fashion that the peptides associate with class I MHC molecules and are presented on the surface of the antigen presenting cell, and consequently are available to stimulate a CTL response.

By preparing the stimulator cells used to generate an in vitro CTL response in different ways, it is possible to control the peptide specificity of CTL response. For example, the CTL generated with a particular peptide will necessarily be specific for that peptide. Likewise, CTL that are generated with a polypeptide or polynucleotide expressing or coding for particular peptides will be limited to specificities that recognize those peptides. More broadly, stimulator cells, and more specifically dendritic cells, can be incubated in the presence of the parent protein. As a further alternative, stimulator cells, and more specifically dendritic cells, can be transduced or transfected with RNA or DNA comprising the polynucleotide sequence encoding the protein. Under these alternative conditions, peptide epitopes that are naturally cleaved out of the protein can associate with an appropriate class I MHC molecule. The selection of antigen presenting cells and the type of antigen with which to stimulate the CTL is left to the ordinary skilled artisan.

In specific embodiments, the methods of the present invention include a method for inducing a CTL response in vitro that is specific for a tumor cell, whereby the method comprises contacting a CTL precursor lymphocyte with an antigen presenting cell that has bound an immunogen comprising one or more of the peptides disclosed according to the invention.

In specific embodiments, the methods of the present invention include a method for inducing a CTL response in vitro, preferably for a cancer stem cell, whereby the method comprises contacting a CTL precursor lymphocyte with an antigen presenting cell that has exogenously acquired an immunogenic oligopeptide or polypeptide that comprise one or more of the peptides disclosed according to the invention.

In specific embodiments, the methods of the present invention include method for inducing a CTL response in vivo, preferably for a cancer stem cell, whereby the method comprises contacting a CTL precursor lymphocyte with an antigen presenting cell that has endogenously acquired an immunogenic oligopeptide or polypeptide that comprise one or more of the peptides disclosed according to the invention.

A yet additional embodiment of the present invention is directed to a process for inducing a CTL response in vitro that is specific for a tumor cell, preferably for a cancer stem cell, comprising contacting a CTL precursor lymphocyte with an antigen presenting cell that is expressing a polynucleotide coding for a polypeptide of the invention and wherein said polynucleotide is operably linked to a promoter.

A yet additional embodiment of the present invention is directed to a process for inducing a CTL response in vivo that is specific for a tumor cell, preferably for a cancer stem cell, comprising contacting a CTL precursor lymphocyte with an antigen presenting cell that is expressing a polynucleotide coding for a polypeptide of the invention and wherein said polynucleotide is operably linked to a promoter.

A variety of techniques exist for assaying the activity of CTL. These techniques include the labeling of target cells with radionuclides such as Na²⁵CrO4 or ³H-thymidine, and measuring the release or retention of the radionuclides from the target cells as an index of cell death. Such assays are well known in the art and their selection is left to the skilled artisan.

Alternatively, CTL are known to release a variety of cytokines when they are stimulated by an appropriate target cell, such as a tumor cell expressing the relevant class I MHC molecule and the corresponding peptide. Non-limiting examples of such cytokines include IFN-gamma, TNFoc, and GM-CSF. Assays for these cytokines are well known in the art, and their selection is left to the skilled artisan. Methodology for measuring both target cell death and cytokine release as a measure of CTL reactivity are given in (Coligan, Jo E. et al, 25 Current Protocols in Immunology, 1999, John Wiley & Sons, Inc., New York).

After expansion of the antigen-specific CTL in vitro, the latter are then adoptively transferred back into the patient, where they will destroy their specific target cell. The utility of such adoptive transfer is demonstrated in (Dudley M. et al. Science 25; 298(5594):850-854 (2002); North, R. J. et al., Infect. Immun., 67:2010-2012, (1999); Ridden, S. R. et al., Science, 257:238-241, (1992)). In determining the amount of cells to reinfuse, the skilled physician will be guided by the total number of cells available, the activity of the CTL as measured in vitro, and the condition of the patient.

Preferably, however, about 1×10⁶ to about 1×10⁴², more preferably about 1×10⁸ to about 1×10⁴⁴, and even more preferably, about 1×10⁹ to about 1×10⁴⁰ peptide-specific CTL are infused. Methodology for reinfusing the T cells into a patient are well known and exemplified in U.S. Pat. No. 4,844,893 to Honski, et al., and U.S. Pat. No. 4,690,915 to Rosenberg.

The peptide-specific CTL can be purified from the stimulator cells prior to infusion into the patient. For example, monoclonal antibodies directed toward the cell surface protein CD8, present on CTL, can be used in conjunction with a variety of isolation techniques such as antibody panning, flow cytometric sorting, and magnetic bead separation to purify the peptide specific CTL away from any remaining non-peptide specific lymphocytes or from the stimulator cells. These methods are well known in the art, and their selection is left to the skilled artisan. It should be appreciated that generation of peptide-specific CTL in this manner obviates the need for stimulating the CTL in the presence of tumor. Thus, there is no chance of inadvertently reintroducing tumor cells into the patient.

Thus, one embodiment of the present invention relates to a process for treating a subject with cancer characterized by cancer stem cells whereby CTL produced in vitro according to the present invention are administered in an amount sufficient to destroy the tumor cells through direct lysis or to effect the destruction of the tumor cells indirectly through the elaboration of cytokines.

Another embodiment of the present invention is directed to a process for treating a subject with cancer characterized by cancer stem cells expressing any class I MHC molecule and an associated epitope, whereby the CTLs are produced in vitro and are specific for the epitope or parent protein and are administered in an amount sufficient to destroy the tumor cells through direct lysis or to effect the destruction of the tumor cells indirectly through the elaboration of cytokines.

Thus, one embodiment of the present invention relates to a process for treating a subject with cancer characterized by cancer stem cells whereby CTLs produced in vitro according to the present invention are administered in an amount sufficient to destroy the tumor cells, and preferably cancer stem cells through direct lysis or to effect the destruction of the tumor cells indirectly through the elaboration of cytokines.

Another embodiment of the present invention is directed to a an immunization process for treating a subject with cancer characterized by cancer stem cells expressing any class I MHC molecule and an associated epitope, whereby the CTLs are produced in vivo and are specific for an epitope or epitopes derived from an oligopeptide or polypeptide administered to the patient in a fashion such that the tumor cells, and preferably cancer stem cells, are destroyed through direct lysis or to effect the destruction of the tumor cells indirectly through the elaboration of cytokines.

In the foregoing embodiments the cancer to be treated may include any cancer, including but not limited to an ovarian carcinoma, breast carcinoma, a colorectal carcinoma, lung carcinoma, heptaocellular carcinoma and prostate carcinoma.

Ex vivo-generated CTL can be used to identify and isolate the T cell receptor molecules specific for the peptide. The genes encoding the alpha and beta chains of the T cell receptor can be cloned into an expression vector system and transferred and expressed in naive T cells from peripheral blood, T cells from lymph nodes, or T lymphocyte progenitor cells from bone marrow. These T cells, which would then be expressing a peptide-specific T cell receptor, would then have anti-tumor reactivity and could be used in adoptive therapy of cancer, and more specifically cancer, colorectal carcinoma, ovarian carcinoma, lung carcinoma, and prostate carcinoma.

In addition to their use for therapeutic or prophylactic purposes, the immunogenic peptides of the present invention are useful as screening and diagnostic agents. Thus, the immunogenic peptides of the present invention, together with modem techniques of gene or antibody screening, make it possible to screen patients for the presence of genes encoding such peptides or proteins from cells obtained by biopsy of tumors detected in such patients. The results of such screening may help determine the efficacy of proceeding with the regimen of treatment disclosed herein using the immunogens of the present invention to target and eliminate cancer cells, preferably cancer stem cells.

Alternatively, the immunogenic peptides disclosed herein, as well as functionally similar homologs thereof, may be used to screen a sample for the presence of CTLs that specifically recognize the corresponding epitopes. The lymphocytes to be screened in this assay will normally be obtained from the peripheral blood, but lymphocytes can be obtained from other sources, including lymph nodes, spleen, tumors, and pleural fluid.

The peptides of the present invention may then be used as a diagnostic tool to evaluate the efficacy of the immunotherapeutic treatments disclosed herein. Thus, the in vitro generation of CTL as described above would be used to determine if patients are likely to respond to the peptide in vivo. Similarly, the in vitro generation of CTL could be accomplished with samples of lymphocytes obtained from the patient before and after treatment with the peptides. Successful generation of CTL in vivo should then be recognized by a correspondingly easier ability to generate peptide-specific CTL in vitro from lymphocytes obtained following treatment in comparison to those obtained before treatment. The oligopeptides of the invention can also be used to prepare class I MHC tetramers which can be used in conjunction with flow cytometry to quantitate the frequency of peptide-specific CTL that are present in a sample of lymphocytes from an individual.

Specifically, for example, class I MHC molecules and associated peptides would be combined to form tetramers as exemplified in U.S. Pat. No. 5,635,363. Said tetramers would find use in monitoring the frequency of CTLs specific for the peptide or peptides in the peripheral blood, lymph nodes, or tumor mass of an individual undergoing immunotherapy with the peptides, oligopeptides, polypeptides, or polynucleotides of the invention, and it would be expected that successful immunization would lead to an increase in the frequency of the peptide-specific CTL.

As stated above, a vaccine in accordance with the present invention may include one or more of the hereinabove described polypeptides or active fragments thereof, or a mixture, composition, or pool, of immunogenic peptides, oligopeptides or polypeptides disclosed herein. When employing more than one polypeptide or active fragment, two or more polypeptides and/or active fragments may be used as a physical mixture or as a fusion of two or more polypeptides or active fragments. The fusion fragment or fusion polypeptide may be produced, for example, by recombinant techniques or by the use of appropriate linkers for fusing previously prepared polypeptides or active fragments.

The immunogenic molecules of the invention, including vaccine compositions, may be utilized according to the present invention for purposes of preventing, suppressing or treating diseases causing the expression of the immunogenic peptides disclosed herein, such as where the antigen is being expressed by tumor cells and in particular by cancer stem cells. As used in accordance with the present invention, the term “prevention” relates to a process of prophylaxis in which an animal, especially a mammal, and most especially a human, is exposed to an immunogen of the present invention prior to the induction or onset of the disease process. This could be done where an individual has a genetic pedigree indicating a predisposition toward occurrence of the disease condition to be prevented. For example, this might be true of an individual whose ancestors show a predisposition toward certain types of cancer.

Alternatively, the immunogen could be administered to the general population as is frequently done for infectious diseases. Alternatively, the term “suppression” is often used to describe a condition wherein the disease process has already begun but obvious symptoms of said condition have yet to be realized. Thus, the cells of an individual may have become cancerous but no outside signs of the disease have yet been clinically recognized. In either case, the term prophylaxis can be applied to encompass both prevention and suppression. Conversely, the term “treatment” is often utilized to mean the clinical application of agents to combat an already existing condition whose clinical presentation has already been realized in a patient.

This would occur where an individual has already been diagnosed as having a tumor. It is understood that the suitable dosage of an immunogen of the present invention will depend upon the age, sex, health, and weight of the recipient, the kind of concurrent treatment, if any, the frequency of treatment, and the nature of the effect desired. However, the most preferred dosage can be tailored to the individual subject, as determined by the researcher or clinician. The total dose required for any given treatment will commonly be determined with respect to a standard reference dose as set by a manufacturer, such as is commonly done with vaccines, such dose being administered either in a single treatment or in a series of doses, the success of which will depend on the production of a desired immunological result (i.e., successful production of a CTL-mediated response to the antigen, which response gives rise to the prevention and/or treatment desired). Thus, the overall administration schedule must be considered in determining the success of a course of treatment and not whether a single dose, given in isolation, would or would not produce the desired immunologically therapeutic result or effect.

The therapeutically effective amount of a composition containing one or more of the immunogens of this invention is an amount sufficient to induce an effective CTL response to the immunogen and to cure or arrest disease progression. Thus, this dose will depend, among other things, on the identity of the immunogens used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Thus, for purposes of prophylactic or therapeutic administration, effective amounts would generally lie within the range of from 1.0 ug to about 5,000 ug of peptide for a 70 kg patient, followed by boosting dosages of from about 1.0 ug to about 1,000 ug of peptide pursuant to a boosting regimen over days, weeks or even months, depending on the recipient's response and as necessitated by subsequent monitoring of CTL-mediated activity within the bloodstream. Of course, such dosages are to be considered only a general guide and, in a given situation, may greatly exceed such suggested dosage regimens where the clinician believes that the recipient's condition warrants more aggressive administration schedule.

Needless to say, the efficacy of administering additional doses, and of increasing or decreasing the interval, may be re-evaluated on a continuing basis, in view of the recipient's immunocompetence (for example, the level of CTL activity with respect to tumor-associated or tumor-specific antigens).

For such purposes, the immunogenic compositions according to the present invention may be used against a disease condition such as cancer by administration to an individual by a variety of routes. The composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time.

Generally, vaccines are prepared as injectables, in the form of aqueous solutions or suspensions. Vaccines in an oil base are also well known such as for inhaling. Solid forms which are dissolved or suspended prior to use may also be formulated. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. These compositions may be sterilized by conventional, well known sterilization techniques including sterile filtration. The resulting solutions may be packaged for use as is, or the aqueous solutions may be lyophilized, the lyophilized preparation being combined with sterile water before administration.

Vaccine compositions may further incorporate additional substances to stabilize pH, or to function as adjuvants, wetting agents, or emulsifying agents, which can serve to improve the effectiveness of the vaccine.

The concentration of the CTL stimulatory peptides of the invention in pharmaceutical formulations necessary to elicit a tumor rejection response are subject to wide variation, including anywhere from less than 0.01% by weight to as much as 50% or more. Factors such as volume and viscosity of the resulting composition must also be considered. The solvents, or diluents, used for such compositions include water, possibly PBS (phosphate buffered saline), or saline itself, or other possible carriers or excipients.

The immunogens of the present invention may also be contained in artificially created delivery vehicles such as liposomes, Immunostimulating Complexes (ISCOMS), slow-releasing particles, and other vehicles that increase the immunogenicity and/or half-life of the peptides or polypeptides in serum. Liposomes include emulsions, foams, micelles, insoluble monolayers, liquid crystals, phospholipid dispersions, lamellar layers and the like. Liposomes for use in the invention are formed from standard vesicle-forming lipids which generally include neutral and negatively charged phospholipids and a sterol, such as cholesterol. The selection of lipids is generally determined by considerations such as liposome size and stability in the blood. A variety of methods is available for preparing liposomes as reviewed, for example, by (Coligan, J. E. et al, Current Protocols in Protein Science, 1999, John Wiley & Sons, Inc., New York) and see also U.S. Pat. Nos. 4,235,871, 4,501,728, 4,837,028, and 5,019,369.

Delivery vehicles containing the peptides or polypeptides of the invention can be directed to the site of lymphoid cells where the vehicles deliver the selected immunogens directly to antigen presenting cells. Targeting can in some cases be achieved by incorporating additional molecules such as proteins or polysaccharides into the outer membranes of said structures, thus resulting in the delivery of the structures to particular areas of the body, or to particular cells within a given organ or tissue. Such targeting molecules may include a molecule that binds to receptor on antigen presenting cells. For example an antibody that binds to CD80 could be used to direct liposomes to dendritic cells.

The immunogens of the present invention may also be administered as solid compositions. Conventional nontoxic solid carriers including pharmaceutical grades of mannitol, lactose, starch, magnesium, cellulose, glucose, sucrose, sodium saccharin, and the like. Such solid compositions will often be administered orally, whereby a pharmaceutically acceptable nontoxic composition is formed by incorporating the peptides and polypeptides of the invention with any of the carriers listed above. Generally, such compositions will contain 10-95% active ingredient, and more preferably 25-75% active ingredient. Aerosol administration is also an alternative, requiring only that the immunogens be properly dispersed within the aerosol propellant. Typical percentages of the peptides or polypeptides of the invention are 0.01%-20% by weight, preferably 1%-10%. The use of a surfactant to properly disperse the immunogen may be required. Representative surfactants include the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride.

Mixed esters, such as mixed or natural glycerides may be employed. The surfactant may constitute 0.1-20% by weight of the composition, preferably 0.25-5%. Typical propellants for such administration may include esters and similar chemicals but are by no means limited to these. A carrier, such as lecithin for intranasal delivery, may also be included.

The peptides and polypeptides of the invention may also be delivered with an adjuvant. Adjuvants include, but are not limited to complete or incomplete Freund's adjuvant, Montanide ISA-51, aluminum phosphate, aluminum hydroxide, alum, and saponin. Adjuvant effects can also be obtained by injecting a variety of cytokines along with the immunogens of the invention. These cytokines include, but are not limited to IL-1, IL-2, IL-7, IL-12, and GM-CSF.

The peptides and polypeptides of the invention can also be added to professional antigen presenting cells such as dendritic cells that have been prepared ex vivo. For example, the dendritic cells could be prepared from CD34 positive stem cells from the bone marrow, or they could be prepared from CD14 positive monocytes obtained from the peripheral blood. The dendritic cells are generated ex vivo using cytokines such as GM-CSF, IL-3, IL-4, TNF, and SCF. The cultured DC are then pulsed with peptides at various concentrations using standard methods that are well known in the art. The peptide-pulsed dendritic cells can then be administered intravenously, subcutaneously, or intradermally, and the immunization may also include cytokines such as IL-2 or IL-12.

The present invention is also directed to a vaccine in which an immunogen of the present invention is delivered or administered in the form of a polynucleotide encoding a polypeptide or active fragment as disclosed herein, whereby the peptide or polypeptide or active fragment is produced in vivo. The polynucleotide may be included in a suitable expression vector and combined with a pharmaceutically acceptable carrier.

For example, the peptides or polypeptides could be expressed in plasmid DNA and nonreplicative viral vectors such as vaccinia, fowlpox, Venezuelan equine encephalitis virus, adenovirus, or other RNA or DNA viruses. These examples are meant to be illustrative only and should not be viewed as self-limiting. A variety of other vectors is available and apparent to those skilled in the art from the description given herein. In this approach, a portion of the nucleotide sequence of the viral vector is engineered to express the peptides or polypeptides of the invention. Vaccinia vectors and methods useful in immunization protocols are described in U.S. Pat. No. 4,722,848, the disclosure of which is incorporated herein by reference in its entirety.

Regardless of the nature of the composition given, additional therapeutic agents may also accompany the immunogens of the present invention. Thus, for purposes of treating tumors, compositions containing the immunogens disclosed herein may, in addition, contain other antitumor pharmaceuticals such as small molecule therapeutics or monoclonal antibodies. The use of such compositions with multiple active ingredients is left to the discretion of the clinician.

In addition, certain immunogens of the present invention can be used to stimulate the production of antibodies for use in passive immunotherapy, for use as diagnostic reagents, and for use as reagents in other processes such as affinity chromatography.

The present invention also relates to antibodies that react with immunogens, such as a polypeptide that comprises within it one or more stretches comprising the sequence of an MHC-associated peptide or oligopeptide. Active fragments of such antibodies are also specifically contemplated. Such antibodies, and active fragments of such antibodies, for example, and Fab structure, may react with, including where it is highly selective or specific for, an immunogenic structure comprising two, three, four or more of the epitopic peptides of the invention, or polypeptides of the invention. Thus, the present invention also relates to a process of using antibodies to elicit an immune response, including the production of protective antibodies, following administration of immunogens of the invention to an immunocompetent animal (i.e., an animal whose immune system has not been compromised, either naturally or experimentally, so as to not exhibit such a reaction, such as a knock-out animal or the like).

With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H2L2) structures with affinity for specific epitopes and antigenic determinants. This has permitted the ready production of antibodies having sequences characteristic of neutralizing antibodies from different species and sources.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab2)′ fragments, capable of reacting with and binding to any of the polypeptides disclosed herein as being receptors. The invention also contemplates the use of very small antibodies, such as ‘nanobodies’ that are capable of specifically binding to antigenic targets.

Regardless of the source of the antibodies, or how they are produced, in vitro or in vivo, using transgenic animals, such as cows, goats and sheep, or using cell culture or using direct chemical synthesis employing no living organisms at any stage of the process, all antibodies have a similar overall three-dimensional structure. This structure is often given as H2L2 and refers to the fact that antibodies commonly comprise two light (L) amino acid chains and two heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarily determining regions” or “CDR” regions. These CDR regions account for the basic specificity of the antibody for a particular antigenic determinant structure.

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

A further embodiment of the present invention relates to a method for inducing a CTL response in a subject comprising administering to subjects an effective (i.e., CTL-stimulating amount) of an immunogen of the invention that does not comprise the entire protein expressing the epitopic peptides disclosed herein (i.e., one that comprises less than the entire source protein where the protein is a naturally occurring polypeptide) in an amount sufficient to induce a CTL response to tumor cells, more specifically against cancer stem cells, thereby eliciting a cellular response against said tumor cells.

A still further embodiment of the present invention relates to a method for inducing a CTL response in a subject, wherein the immunogen is in the form of a polynucleotide. In one non-limiting example, the method comprises administering to subjects at least one CTL epitope, wherein said epitope or epitopes are selected from a group comprising the peptides disclosed according to the invention, and are coded within a polynucleotide sequence that does not comprise the entire protein coding region, in an amount sufficient to induce a CTL response to tumor cells, more specifically in cancer stem cells.

CSC may be isolated from primary tumor tissues, pleural effusions (Al-Hajj, M., M. S. Wicha, A. Benito-Hemandez, S. J. Morrison, and M. F. Clarke. Proc Natl Acad Sci USA 100:39832 (2003.)) or from established breast cancer cell lines, such as SKBR3, MDA 231, and MCF7 (Ponti, D., A. Costa, N. Zaffaroni, G. Pratesi, G. Petrangolini, D. Coradini, S. Pilotti, M. A. Pierotti, and M. G. Daidone. Cancer Res 65:55063 (2005.)). Isolated CSC are characterized by flow cytometry using cell surface markers appropriate for each tumor type and known to those skilled in the art. The cells may be expanded in vitro or in SCID mice for MHC peptide analysis. CD34+ stem cell populations can be obtained from GSF-mobilized healthy human peripheral blood, cord blood or other such sources, and expanded if necessary with cytokines to obtain 10⁸ cells using procedures known to those skilled in the art. Cell populations can comprise any HLA type.

As a nonlimiting example, human breast cancer patient malignant effusions may be obtained by subjecting the fluid to ficol separation to remove debris and red blood cell contamination. Typical yields of cells range from 200 million to 2 trillion cells per liter of fluid. For tumor cell lines, the cells are cultured in RPMI 1640 medium supplemented with 10% FCS, pen-strep-glutamine, or other suitable media. Antibody-labeled cells may be isolated by FACS sorting or alternatively by using antibody-coated magnetic beads. Flow cytometric staining for breast tumor stem cell phenotypic analysis is used to identify lineage negative (CD2, 3,10,16,18,31,45, 64,140b), CD44+-FITC labeled antibody/CD24lo PerCP labeled antibody from human breast cancer patient pleural effusions. For breast tumor cell lines, analysis does not require the extensive array of lineage negative markers, since there is no blood cell contamination of these samples and simply using CD44+, CD24-/lo is sufficient. The first step in cell sorting of CSC populations from malignant effusions is to use antibodies to deplete the lineage markers (CD2, 3, 10,16,18,31, 45, 64,140b), to greatly reduce the number of non-CSC cells and thereby reduce the cell sorting time. This is followed by sorting of the cells based on markers for CD44+/CD24lo. Sorted cells may then be cultured in vitro or alternatively used for in vivo experiments by implantation into SCID mice.

CD34 positive cells are purified from GSF mobilized PBL or cord blood using anti-CD34 antibody coated magnetic beads to obtain purified CD34+ cells and then cell sorted to obtain the CD38neg fraction. In vitro expansion is performed using established methods (Lam, A. C., K. Li, X. B. Zhang, C. K. Li, T. F. Fok, A. M. Chang, A. E. James, K. S. Tsang, and P. M. Yuen. Transfusion 41:1567 (2001); Li, K., C. K. Li, C. K. Chuen, K. S. Tsang, T. F. Fok, A. E. James, S. M. Lee, M. M. Shing, K. W. Chik, and P. M. Yuen. Eur J Haematol 74:128 (2005)).

Expansion (10⁸-10⁹) is performed either in cell culture or in SCID mice, for example, (Al-Hajj et al. Proc Natl Acad Sci 100:3983 (2003)). If cell lines are expanded using cell culture, their tumorigenicity may additionally be validated in vivo by implantation in SCID mice using methods known to those skilled in the art. At the end of the expansion period, the percentage of cells retaining the cancer stem cell phenotype is assessed.

MHC peptide complexes are isolated from cells using immunoaffinity purification. First, cell lysates are prepared from by homogenization and freeze/thaw in buffer containing 1.0% NP40; preferably about 1-5×10⁸ cells are used. The lysates are cleared by centrifugation at 2000 rpm for 30 minutes to remove the cell debris. MHC/peptide complexes are isolated by immunoaffinity chromatography using W632 (monoclonal antibody recognizing pan MHC class I molecule) antibody coated protein A/G beads (UltraLink Immobilized Protein A/G, Pierce, Rockford, Ill.) or equivalent, to isolate pan MHC Class I/peptide complexes. Protein A/G beads are washed with low pH buffer followed by PBS rinses. The beads are then incubated with 0.5 mg W632 antibody at room temperature for 2 hours. Labeled beads are washed three times to remove unbound antibodies. The antibody-coated beads are added to the prepared cell lysate. The mixture is incubated for two hours at room temperature with continuous rocking. The beads are separated from the lysate by centrifuging at 1000 rpm for 5 minutes. The recovered lysate is incubated for an additional 2 hours at room temperature with another set of prepared antibody coated Protein A/G beads. The bound MHC complexes are eluted from the beads by the addition of 0.1% Trifluoroacetic acid, (TFA), pH 1.5, 200 μl of TFA, the beads are centrifuged, and the supernatant removed. This process is repeated three times to give a final volume of 600 μl. The eluate is then boiled for 5 minutes to denature the proteins, after which, it is dried down to almost dryness using a speed-vac and reconstituted to 50 μl or appropriate volume with SEC running buffer.

Peptide mixtures are purified to eliminate any components that may have non-specifically bound to the antibodies during the aforementioned purification step. Fractions are first reduced to one half their volumes using vacuum centrifugation without heat. They are then diluted to 100 microliters with 5% triethylamine in 50/50 isopropyl alcohol/water. This mixture is placed into a separate 0.1 micron spin filter containing approximately 10 micrograms of isothiocyanato glass (DITC) and allowed to incubate for 45 minutes at 45° C. with gentle shaking. Only chemical molecules containing primary amines, such as the N-terminus of a peptide, react and covalently bind to the DITC-derivatized glass. All filter units then undergo centrifugation at 10,000 rpm for 2 minutes. The filtrate volume containing the unbound material is discarded. The DITC glass is washed several times with organic and ionic buffers at neutral pH; the wash solution is filtered and discarded. Finally, the peptides are released from the DITC glass by the addition of 100 microliters of TFA with incubating for 10 minutes at 45° C. with gentle shaking. The filter unit is then spun at 10,000 rpm for 2 minutes to separate the peptides from the DITC glass, the filtrate, containing the peptides, are collected and saved.

The purified peptide mixture first undergoes size exclusion chromatography (SEC) in order to separate any proteins from the peptides. SEC is performed on an ABI 140B LC system flowing at 250 ul/min in isocratic mode with an ABI 785A UV detector monitoring at 254 nm. A Zorbax GF-250 4.6×250 mm (Agilent, Palo Alto, Calif.) column is used with a mobile phase consisting of 1% acetic acid (˜pH 3.5)+10% acetonitrile. The peptide-containing fraction is collected from 14 minutes to 18 minutes as determined by a calibration mix injection. The peptide-containing fraction then undergoes a first dimension fractionation via HPLC. A 1 mm×250 mm PLRP-S 100A° Polymer column is then used with an ABI 140B LC system flowing at 50 ul/min in gradient mode with an ABI 785A UV detector monitoring at 214 nm. Solvent A is 2% ACN in H₂O+0.1% TFA and solvent B is 80% ACN in H₂O+0.09% TFA using a gradient of 2% B to 60% B in 60 minutes. Fractions are collected at one-minute intervals and further processed for mass spectrometry analysis.

Peptides are sequenced by the technique of Collision Activated Dissociation (CAD) on an ion trap mass spectrometer equipped with on-line microcapillary HPLC and a microspray ionization source. Each HPLC fraction is loaded onto the microcapillary HPLC column, fractionated, and eluted directly into the electrospray ionization source. The microcapillary column (75 um×100 mm) is packed with C-18 separation resin. Solvent A is 2% ACN in H₂O+1% acetic acid and solvent B is 80% ACN in H₂O+1% acetic acid, using a gradient of 2% B to 60% B in 240 minutes at a flow rate of 200 nl/min achieved via splitting flow from a Eldex micro pumping system (Eldex, Napa, Calif.). The mass spectrometer is configured such that a full mass scan (spectra) is acquired about every 10 seconds. From the spectra, the computer selects the 5 most abundant peaks and performs a CAD scan on each of the 5 peaks. Over the course of 4 hours, for a single HPLC fraction, approximately 1400 full scans and 7000 CAD scans are acquired.

CAD spectra are searched against databases using search program software in order to interpret the data and derive peptide sequences. The reduced spectrum sets are then be searched against a non-redundant human genomic database using SeQuest, Mascot and Scaffold software. Results are returned as a confidence score, which is then be manually verified. CAD mass spectra are used to search various genomic databases to find potential sequences for the candidate peptides and to identify the protein of origin of these peptides. The search results are assembled as a dataset for comparison studies.

While the examples below are provided to illustrate the invention, it is to be understood that these methods and examples in no way limit the invention to the embodiments described herein and that other embodiments and uses will no doubt suggest themselves to those skilled in the art. All publications, patents, and patent applications cited herein are hereby incorporated by reference, as are the references cited therein. It is also to be understood that throughout this disclosure where the singular is used, the plural may be inferred and vice versa and use of either is not to be considered limiting.

### EXAMPLE 1

Cell Lines

MDA-mb-231 (HLA-A2, A24), a mammary gland ductal carcinoma cell line established from a pleural effusion, was obtained from ATCC (Manassas, Va.) and cultured according to the ATCC protocol. The cell line SKOV3.A2 is an HLA-A2.1 transfectant of the original ATCC (Manassas, Va.) ovarian adenocarcinoma line SKOV3 (HLA-A3, 68, B18, 35, Cw5, -) and was obtained from the M. D. Anderson Cancer Center, Houston, Tex. A second ovarian cancer cell line OVCAR3 (HLA-A2, 29 B7, 58) was procured from ATCC. Both cell lines were cultured according to methods described in Ramakrishna, V. et al. 2003 International Immunology 15(6):751-763.

### EXAMPLE 2

Immunoaffinity Purification

All tumor lines were maintained in RPMI 1640 medium containing 10% heat-inactivated FBS, 2 mM L-glutamine, 10 mM HEPES, penicillin (100 U/ml)-streptomycin (50 μg/ml) solution and 1% sodium pyruvate solution (all from Sigma, St Louis, Mo.). The SKOV3.A2 cell line was continuously maintained in 250 μg/ml G418 (Invitrogen). The cells were harvested by treatment with 0.45% trypsin and 0.32 mM EDTA, washed two times in phosphate-buffered saline solution (pH 7.4), and stored as cell pellets at −80° C. Aliquots of 6-8×10¹⁰ cells were solubilized at 5-10×10⁶ cells/ml in 20 mM Tris, pH 8.0, 150 mM NaCl, 1% CHAPS, 18.5 μg/ml iodoacetamide, 5 μg/ml aprotonin, 10 μg/ml leupeptin, 10 μg/ml pepstatin A, 5 mM EDTA, 0.2% sodium azide, and 17.4 μg/ml phenylmethylsulfonyl fluoride for 1 h. This and all subsequent steps were performed with ice-cold solutions and at 4° C. The lysates were then centrifuged at 100,000×g, the pellets discarded, and the supernatants passed through a 0.22 μm filter. The supernatants were then passed over a series of columns with the first containing Sepharose, and the second containing the HLA-A1-specific monoclonal antibody, GAP-A1, bound to a protein A-Sepharose matrix. The second column was then sequentially washed with 20 column volumes of 20 mM Tris, pH 8.0, 150 mM NaCl, 20 column volumes of 20 mM Tris, pH 8.0, 1.0 M NaCl, and 20 column volumes of 20 mM Tris, pH 8.0. The peptides were eluted from the column with 5 column volumes of 10% acetic acid. The isolated HLA-A1 molecules were then boiled for 5 min to further dissociate any bound peptide from the heavy chains. The peptides were then separated from the co-purifying class I heavy chain and β₂-microglobulin by centrifugation on an Ultrafree-CL membrane with a nominal molecular weight cut-off of 5,000 Daltons (Millipore, Bedford, Mass.).

OVCAR3 or SKOV3 cells were prepared using the same procedure as just described except that HLA-A2 molecules were prepared using HLA-A2-specific antibodies.

### EXAMPLE 3

Peptide Fractionation

The peptide extracts were fractionated by RP-HPLC (Reversed Phase-High Performance Liquid Chromatography) using an Applied Biosystems (ABI) model 140B system. The extracts were concentrated by vacuum centrifugation from about 20 ml down to 250 μl and injected into either a Brownlee (Norwalk, Conn.) C₁₈ Aquapore column (2.1 mm×3 cm; 300 Å; 7 μm) or a Higgins (Mountain View, Calif.) C18 Haisil column (2.1 mm×4 cm; 300 Å; 5 μm). The peptides were eluted by first using a gradient of acetonitrile/0.085% TFA (trifluoroacetic acid) in 0.1% TFA/water, with the concentration of acetonitrile increasing from 0-9% (0-5 minutes), 9-36% (5-55 minutes), and 36-60% (55-62 minutes). A second dimension fractionation of combined fractions 17 and 18 from the first dimension (TFA) fraction was accomplished using the same gradient but with the substitution of HFBA (heptafluorobutyric acid) for TFA. The flow rate was 200 μl/min, and fractions were collected at 1 min (Brownlee column) or 40 second (Higgins column) intervals. A third dimension of RP-HPLC was achieved using an Eldex (Napa, Calif.) MicroPro Pump, a homemade C₁₈ microcapillary column, and an ABI model 785A UV absorbance detector. The column was prepared by packing a 27 cm bed of 10 μm C₁₈ particles in a section of 285 μm o.d./75 μm i.d. fused silica (Polymicro Technologies, Phoenix, Ariz.). Peptides in combined fractions 26 and 27 of the second dimension fraction were loaded onto this column and eluted with a gradient of acetonitrile/0.67% triethylamine acetate/water in 0.1% triethylamine acetate/water, with the concentration of acetonitrile increasing from 0-60% in 40 minutes. The flow rate was about 300 nl/min, and fractions were collected into 25 μl of water every 30 sec. In all RP-HPLC experiments, peptides were detected by monitoring UV absorbance at 214 nm.

### EXAMPLE 4

Mass Spectrometric Analysis

The second dimension HPLC fraction was analyzed using an affluent splitter on the microcapillary HPLC column. In this experiment, the column (360 μm o.d.×100 μm i.d. with a 25 cm C₁₈ bed) was butt connected with a zero dead volume tee (Valco, Houston, Tex.) to two pieces of fused silica of different lengths (25 μm and 40 μm i.d.). Peptides were eluted with a 34 min gradient of 0-60% acetonitrile. The 25 μm capillary deposited one-fifth of the HPLC effluent into the wells of a microtiter plate for use in CTL epitope reconstitution assays, whereas the remaining four-fifths of the effluent was directed into the mass spectrometer. Ions were formed by electrospray ionization, and mass spectra were recorded by scanning between mass to charge ratios (m/z) 300 and 1400 every 1.5 seconds. Peptide sequences were determined by CAD (collision-activated dissociation) tandem mass spectrometry as described in the literature (Hunt, D. F. et al., Proc. Natl. Acad. Sci. U.S.A, 83:6233-6237, (1986)).

### EXAMPLE 5

Homology Searches of Identified Peptide Sequences

Proteins containing peptides corresponding to the masses identified by MS were analyzed with the search algorithm, SEQUEST. Searches were carried using SwissProt, a curated human protein database http://www.expasy.org/sprot/ to identify MHC-associated peptides and their respective source proteins that have been identified in various types of stem cells (Table 2). Table 3 describes SEQ ID NO: 1-241, which are MHC-associated peptides (active fragments) isolated from MDA-mb-231, Ovcar2 or SKOV3.A2 tumor cells. Table 4 lists SEQ ID NO: 799-1039, the original parent proteins from which MHC-associated peptides (active fragments) were identified in one or more of the three cancer cell lines MDA-mb-231, Ovcar3 or SKOV3.A.2.

TABLE 2
Identification of Tumor Cell Antigens also expressed in human stem cells

| | | | Reference |
|---|---|---|---|
| | | | identification |
| | | Motif | and peptide |
| Source Protein | Stem Cell Type¹/Tumor association | Rank² | sequence |
| Macrophage | hMSC/upregulation by TNF associated | 1 | NF00100873 |
| Migration | with tumor growth | | P14174 |
| Inhibitory | | | FLSELTQQL |
| Factor | | | |
| MCT-1 | Lymphomas, epithelial breast tumors/ | 1 | NF00091791 |
| protein | cell-cycle progression associated | | Q9ULC4 |
| | oncogene; expressed in mammary | | YLNDGLWHM |
| | epithelial tumors | | |
| DEAD/H Asp-Glu- | hESC/RNA processing; hedgehog | 1 | NF00130810 |
| Ala-Asp/His) box | pathway associated; c-myc | | Q96FC9 |
| polypeptide 11 | associated | | QLAQFVHEV |
| Bone morphogenetic | hESC, hMSC/tgf-beta family serine- | 5 | NF00100412 |
| protein receptor | threonine kinase; occurs in epithelium; | | Q13873 |
| type II precursor | associated with ↑ tumor growth | | SLDERPVAV |
| Protocadherin gamma | hESC and progenitors/possible role in | 3 | NF00117260 |
| A6 precursor | cell survival or cell adhesion | | O60330 |
| (PCDH-gamma-A6) | | | FLQTYSHEV |
| Retinoblastoma- | hESC/cell cycle regulation; Shh | Not | NF00122941 |
| associated protein | suppression of Rb may stimulate | predicted | Q9UK61 |
| (RAP 140) | meduloblastoma cell proliferation | | FLFQEPRSI |
| Vimentin | hESC,hMSC/gynecologic tumor marker | Not | NF00074186 |
| | | predicted | P08670 |
| | | | DAINTEFKNTR |
| T-plastin | hEpSC, hMSC/tgf-beta regulated, assoc | 5 | NF00123528 |
| | with actin filament organization; | | P13797 |
| | lymphoma marker | | NLFNKYPAL |
| Notch protein | hHSC, hNSC/self-renewal in hHSC and | NR³ | NF00094242 |
| homolog TAN-1 | lineage-specific differentiation; | | P46531 |
| precursor | role in T cell development, down- | | NGGTCEDGIN |
| | regulation in cervical carcinoma cell | | |
| | line inhibited proliferation in | | |
| | vitro and tumorigenicity in vivo | | |
| Bax inhibitor-1 | hHSC, hNSC/BCL-2 family, role in | NR | NF00134091 |
| | apoptosis, neural SC development; | | P55061 |
| | downregulated in endometrial carcinoma | | AYVHMVTHF |
| | (↑proliferation) | | |
| F box/WD repeat | HESC, hNSC, hHSC/Family with WD motifs; | | NF00075424 |
| protein 8 | roles in cell cycle progression, signal | | Q9P2S5 |
| | transduction, apoptosis, and gene | | TEFGHIPAAI |
| | regulation | | |
| Protem-tyrosine | hHSC, D. melanogaster germinal sc/TNF- | To long | NF00117313 |
| kinase JAK1 | alpha activated; assoc with stem cell | for | P23458 |
| | self-renewal; assoc with B cell tumors; | algorithm | KMIGPTHGOQMTV |
| | JAK/STAT deregulation associated with | | |
| | multiple human tumors | | |
| Dj-1 | Fibroblasts/regulates PTEN tumor | NR | NF01304682 |
| | suppressor; circulating breast tumor | | Q99497 |
| | marker; epithelialization gene | | HEIGFGSKV |
| Keratin | hMSC, hEpSC/breast tumor marker; | NR | NF01616723 |
| 8/cytokeratins | epithelial tumor-associated | | P05787 |
| 8 type II cyto- | | | GKLVSESSDVLPK |
| skeletal 8 | | | |
| Lactadherin | hHSC/mammary-epithelium protein, | NR | NF00128029 |
| precursor (Milk | breast tumor marker; associated with | | Q80431 |
| fat globule-EGF | metastasis in disseminated cancer | | HEYLKAFKV |
| factor 8) (Breast | cells in haemopoietic compartments | | |
| epithelial antigen | such as bone marrow or in lymph | | |
| BA46) | nodes | | |

¹hESC, human embryonic stem cells; hHSC human hematopoietic stem cells; hMSC, human mesenchymal stem cells; hNSC, human neural stem cells; hEpSC human epithelial stem cells
²MHC binding affinity calculation for the specific protein, ranked according to the algorithm of Parker et al. J. Biological Physics 28 (2) 183-194 (2004).
³Not Predicted (more than 10 amino acids in length)
⁴Not Ranked (peptide not present in the top 20 rank)

### EXAMPLE 6

Peptide Synthesis

Peptides were synthesized using a Gilson (Madison, Wis.) AMS 422 multiple peptide synthesizer. Quantities of 10 μMol were synthesized using conventional FMOC amino acids, resins, and chemical techniques. Peptides were purified by RP-HPLC using a 4.6 mm×100 mm POROS (Perseptive Biosystems, Cambridge, Mass.) column and a 10 min, 0-60% acetonitrile in 0.1% TFA gradient.

### EXAMPLE 7

Generation of Monocyte-derived Dendritic Cells and Peptide Loading

PBMC were purified from HLA-A2⁺ normal donor blood using lymphocyte separation media (Cappel ICN Biomedical, Aurora, Ohio). PBMC (5.3×10⁶) were added to individual wells of a 24-well cluster plate (Costar, Coming, N.Y.) in 1.0 ml of serum-free AIM-V medium (Life Technologies) and allowed to adhere for 60 min at 37° C. Non-adherent cells were removed and saved as a source of effector T cells. Adherent PBMC (˜8.3×10⁵/well) were then pulsed with 50 mg/ml synthetic peptides in serum-free AIM-V medium containing 1.5 mg/ml β₂-microglobulin (Calbiochem-Novabiochem, San Diego, Calif.) and incubated for 2 h at 37° C. Unbound peptides were aspirated and the wells washed with media.

Monocyte-derived DC were generated as follows. PBMC (5.3×10⁷) were allowed to adhere in T-75 flasks (Corning) in 10 ml of serum-free AIM-V medium for 60 min at 37° C. Non-adherent cells were collected as a source of effector T cells and pooled with the previous collection above. Adherent monocytes in flasks were then exposed to recombinant human granulocyte macrophage colony stimulating factor (GM-CSF, 25 ng/ml; Peprotech) and recombinant human IL-4 (100 ng/ml; Peprotech) in 10 ml of AIM-V medium containing 10% heat-inactivated FBS. DC obtained by this method [immature DC (iDC)] are characterized by expression of low levels of CD83, CD80, CD86, and HLA class I and class II molecules (data not shown).

Mature DC (mDC) were obtained by exposing day 5 DC cultures to recombinant soluble CD40 ligand (sCD40L; Peprotech) at 1.5 mg/ml for 24 h in the presence of 25 ng/ml GM-CSF and are characterized by expression of high levels of CD80, CD86, and HLA class I and class II molecules. mDC were harvested, washed, pulsed with 5 mg/ml peptide in serum-free AIM-V medium and irradiated (5000 rad) prior to use as stimulators.

### EXAMPLE 8

Generation of Peptide-specific CTL

The protocol used here is a modification of the method described by Plebanski et al. (Eur. J. Immunol. 25:1783, (1995)). CTL to peptide were generated by 3±4 cycles of stimulation with peptide-loaded APC. For the first round of stimulation (day 0), T cells or non-adherent PBMC from above (2.3×10⁶/ml or 4.3×10⁶ per well) were added in bulk (CD4⁺, CD8⁺, NK, etc.) to adherent PBMC-loaded peptides in serum-free medium (50 mg/ml), β₂-microglobulin (1.5 mg/ml) (Calbiochem-Novabiochem), recombinant human IL-7 (5 ng/ml) (Peprotech) and keyhole limpet hemocyanin (5 mg/ml) (Sigma, St Louis, Mo.). Cultures were re-stimulated with iDC every 7 days, pulsed with varying amounts of peptide (second round 25 mg/ml, third round 10 mg/ml) and irradiated (5000 rad) on day 8. At each re-stimulation, the T cells were transferred to new plates by first aspirating 70% of spent media in wells and then transferring the pooled contents to a new plate. Fresh IL-7 was added at each re-stimulation. The responder:stimulator (T cell:DC) ratio was set at 20 for each stimulation. Recombinant human IL-2 (10 U/ml) was added on day 5 after each re-stimulation.

Prior to ⁵¹Cr-release assay, the T cells were harvested and CD8⁺ T cells were purified by positive selection using CD8⁺ microbeads immunomagnetic cell separation with MACS kit (Miltenyi Biotec, Auburn, Calif.). If a fourth round of stimulation was necessary following CTL analysis, the CTL were pulsed as before, except with 5±10 mg/ml of peptide.

### EXAMPLE 9

Generation of Allospecific CTL

HLA-A2-allospecific CTL were obtained in a mixed lymphocyte reaction by repeated stimulation of HLA-A3⁺ PBMC (responders) with irradiated HLA-A2⁺ stimulator PBMC at a ratio of 10:1 in the presence of 10 U/ml IL-2. Stimulation was repeated weekly with PBMC from different HLA-A2⁺ donors so as to minimize alloresponse to non-HLA-A2 antigens. T cells were assessed for lysis on several HLA-A2⁺ targets including tumor cells, EBV-B cells and HLA-A3⁺ targets every week after the third round of stimulation.

### EXAMPLE 10

Cytotoxic T Lymphocyte Expansion

Expansion of large numbers of peptide-specific or HLA-A2-allospecific CTL was achieved by culturing 5.3×10⁴±1.3×10⁵ T cells around day 6 or 7 post peptide- or allostimulation in the presence of 2.5-3.0×10⁷ irradiated (5000 rad) allogeneic normal donor PBMC coated with anti-CD3 antibody at 10 ng/ml (BD PharMingen, San Diego, Calif.) and 25 U/ml of recombinant human IL-2 (Peprotech) in a final volume of 30 ml RPMI medium. Media changes with IL-2 addition (50 U/ml) were effected on days 5 and 8. Cells were harvested for cytotoxicity assays on days 10±12 and re-stimulated or frozen for later use.

### EXAMPLE 11

Interferon-γ Enzyme-linked Immunospot (ELISPOT) Assay

MultiScreen-IP opaque 96-well plates (High Protein Binding Immobilon-P membrane, Millipore, Bedford, Mass.) were coated overnight at room temperature with 50 μl/well of 20 μg/ml mouse anti-human IFN-γ mAb (BioSource, Camarillo, Calif.) in DPBS (Invitrogen). After overnight incubation, the plates were washed three times with 200 μl DPBS/well and blocked with 200 μl/well of RPMI 1640, 10% human AB serum (Mediatech, Herndon, Va.), 25 mM Hepes, 2 mM L-glutamine, 100 U/ml penicillin and 100 μg/ml streptomycin (Invitrogen) for 2 h at 37° C., 5% CO₂. Harvested effector antigen specific CTL were added to the plates in triplicates at 1×10⁵ cells/well with either culture medium alone or tumor target cells at various effector:target ratios and cultured for 48 h at 37° C., 5% CO₂. The plates were washed manually 6 times with 200 μl/well of DPBS/0.05% Tween. 50 μl/well of 2 μg/ml mouse anti-human IFN-γ-biotinylated mAb (BD Pharmingen, San Diego, Calif.) in DPBS/1% BSA/0.05% Tween was added and the plates were incubated for 2 h at room temperature. Plates were manually washed 4 times with DPBS and 50 μl/well of streptavidin HRP (BD Pharmingen) diluted 1:2000 in DPBS/1% BSA was added for 1 h at room temperature. The plates were washed a final 4 times with DPBS. Spots were visualized by adding 100 μl/well of TrueBlue™ Peroxidase Substrate (KPL, Gaithersburg, Md.) for 2 min. Plates were scanned and counted using the ImmunoSpot analyzer (Cellular Technology, Ltd., Cleveland, Ohio) to determine the number of spots/well. The precursor frequency of tumor-specific T cells was determined by subtracting the background spots in tumor alone and PBMC alone from the number of spots seen in response to tumor cells.

### EXAMPLE 12

Preparation and Administration of an Immunotherapeutic Peptide Formulation to Cancer Patients

For each patient cohort, the study drug consists of one or more CTL-reactive tumor rejection peptides identified in Example 11, with 100 mcg of each peptide mixed with 1 ml Montanide ISA 51 and 100 mcg GM-CSF. Additionally, a second cohort receives a study drug consisting of one or more peptides identified in Example 11 with 1 mg of each of the peptide mixed with 1 ml Montanide ISA 51 and 100 mcg GM-CSF. On the day of immunization, each vial of peptides is thawed, mixed with 100 microliters of saline and drawn up into a 3 ml syringe and emulsified in Montanide ISA 51 (1 ML) and mixed with GM-CSF for a total volume of 2 ml. The mixture is injected intradennally for the first 200 microliters and then the remainder is injected subcutaneously.

TABLE 3

Description of Active Fragments, Parent Sequence
Identification and Parent SwissProt Identification
Number for peptides 1-125.

| | Peptide | | SwissProt |
|---|---|---|---|
| SEQ ID NO: | Fragment | Parent Protein Identification | ID No. |
| SEQ ID NO:1 | NMEEQPINI | Transcriptional repressor CTCF | P49711 |
| SEQ ID NO:2 | FLASSSAEEQ | Timeless homolog | Q9UNS1 |
| SEQ ID NO:3 | VKLPDGYEF | Galectin-1 | P09382 |
| SEQ ID NO:4 | SGVDFLIFQV | Bone specific GMF608 | AAQ16156.1 |
| SEQ ID NO:5 | VGAAFAMPLPP | Amelogenin | Q99218 |
| SEQ ID NO:6 | FIVDPVSNL | Desmoplakin | P15924 |
| SEQ ID NO:7 | CAWNPVSDL | Nuclear receptor co-repressor/ | Q9BZK7 |
| | | HDAC3 complex subunit TBLR1 | |
| SEQ ID NO:8 | TCLAINSCALG | MEGF6 | O75095 |
| SEQ ID NO:9 | IADFGLAKLL | Tyrosine-protein kinase JAK3 | P52333 |
| SEQ ID NO:10 | HSDEGGVASL | Trophinin-associated protein | Q12815 |
| | | (Tastin) | |
| SEQ ID NO:11 | NMTVLSPYL | Adenomatous polyposis coli | P25054 |
| | | protein | |
| SEQ ID NO:12 | YVKDIYAYL | G2/mitotic-specific cyclin B1 | P14635 |
| SEQ ID NO:13 | TLAKYLMEL | G2/mitotic-specific cyclin B1 | P14635 |
| SEQ ID NO:14 | VQPVTPEPCV | Semaphorin 5B precursor | Q9P283 |
| SEQ ID NO:15 | VIIDQLPDL | LDL receptor-related protein 6 | O75581 |
| SEQ ID NO:16 | SLFPGKLEVV | Flightless-I protein homolog | Q13045 |
| SEQ ID NO:17 | TLPEAIHFL | Flightless-I protein homolog | Q13045 |
| SEQ ID NO:18 | YLPELLQTV | Ectoderm-neural cortex-1 | O14682 |
| | | protein (ENC-1) | |
| SEQ ID NO:19 | TNVTSLLKTV | Talin 1 | Q9Y490 |
| SEQ ID NO:20 | WAKATLLQGG | Semaphorin 6B precursor | Q9H3T3 |
| | | (Semaphorin Z) | |
| SEQ ID NO:21 | SLQEEIAFL | Vimentin | P08670 |
| SEQ ID NO:22 | NLAEDIMRL | Vimentin | P08670 |
| SEQ ID NO:23 | ILLAELEQL | Vimentin | P08670 |
| SEQ ID NO:24 | MVLKAKFQV | Galectin-8 | CAA62904.1 |
| SEQ ID NO:25 | LALAALPAAL | Wnt-1 proto-oncogene protein | P04628 |
| | | precursor | |
| SEQ ID NO:26 | YGLPVVVKL | Beta-catenin | P35222 |
| SEQ ID NO:27 | GLLGTLVQL | Beta-catenin | P35222 |
| SEQ ID NO:28 | ENAGTEEIKN | Programmed cell death 4 | NP_663314.1 |
| | | isoform 2 | |
| SEQ ID NO:29 | EALLGVPLI | Lunatic fringe | AAC51360.1 |
| SEQ ID NO:30 | RLPDIFEAQI | Keratin, type II cyto- | P08729 |
| | | skeletal 7 | |
| SEQ ID NO:31 | YYISHSPQA | Ephrin A1 isoform b | NP_872626.1 |
| | | precursor | |
| SEQ ID NO:32 | VLIPKLPQL | ORMDL3 | Q8N138 |
| SEQ ID NO:33 | KPETFEHLF | Leptin receptor precursor | P48357 |
| | | (LEP-R) | |
| SEQ ID NO:34 | LDLIMKRME | Ras-related protein Rab-27A | P51159 |
| SEQ ID NO:35 | EALNKKAIQI | FKBP-rapamycin associated | P42345 |
| | | protein (FRAP) | |
| SEQ ID NO:36 | ISNDKFEYL | Transcription factor Dp-1 | Q14186 |
| SEQ ID NO:37 | RHPGPGSFGR | Epbrin-B3 precursor | Q15768 |
| SEQ ID NO:38 | TVTDFLAIDA | Semaphorin 6A precursor | Q9H2E6 |
| SEQ ID NO:39 | VEMANLLNL | Homeobox protein Hox-B3 | P14651 |
| SEQ ID NO:40 | RLYYIGGEVF | SMAD 2 | Q15796 |
| SEQ ID NO:41 | RLPPPFPGL | Sorting nexin 1 | Q13596 |
| SEQ ID NO:42 | IMKVGQDPN | Ephrin-B1 precursor | P98172 |
| SEQ ID NO:43 | ILVTLTQLI | Eyes absent homolog 1 | Q99502 |
| SEQ ID NO:44 | NDVMIRKEA | Mago nashi protein homolog | P50606 |
| SEQ ID NO:45 | KILSTHLL | hCLOCK | O15516 |
| SEQ ID NO:46 | SELQVLLGF | Protein inhibitor of activated | Q9Y6X2 |
| | | STAT protein 3 | |
| SEQ ID NO:47 | TLFTVLTYL | Frizzled 7 precursor | O75084 |
| SEQ ID NO:48 | PMCETLTYI | Zinc finger protein 294 | O94822 |
| SEQ ID NO:49 | AVIVLVALL | Leukosialin precursor CD34 | P16150 |
| SEQ ID NO:50 | VMLDVPIRL | Ras GTPase-activating protein | Q9UJF2 |
| | | nGAP | |
| SEQ ID NO:51 | ILLKDILSV | Serine-protein kinase ATM | Q13315 |
| SEQ ID NO:52 | VTGLCTLFTL | Smoothened homolog precursor | Q99835 |
| SEQ ID NO:53 | SILSLVTKI | NF45 protein | Q12905 |
| SEQ ID NO:54 | KLLLRLPSL | COUP transcription factor 2 | P24468 |
| SEQ ID NO:55 | SVIEQLFFV | COUP transcription factor 2 | P24468 |
| SEQ ID NO:56 | ALQTMEPALP | Disheveled associated activator | Q9Y4D1 |
| | | of morphogenesis 1 | |
| SEQ ID NO:57 | DAIIIGEGAQM | Wnt-7b protein precursor | P56706 |
| SEQ ID NO:58 | ATSQGLLIR | Semaphorin 3C precursor | Q99985 |
| SEQ ID NO:59 | TVLSNNLSPF | E1A-associated protein p300 | Q09472 |
| SEQ ID NO:60 | AVIENPEML | HIRA protein | P54198 |
| SEQ ID NO:61 | KIDNSLDKL | Activin | Q04771 |
| SEQ ID NO:62 | GGKMLLIAIL | Angiopoietin 1 | Q02763 |
| SEQ ID NO:63 | MLPSILNQL | Transcription factor BTF3 | P20290 |
| SEQ ID NO:64 | APQNLIFNI | Ephrin type-A receptor 7 | Q15375 |
| SEQ ID NO:65 | ISHEQLPEL | Bone morphogenetic protein 10 | O95393 |
| SEQ ID NO:66 | ENNYTALQN | Fibroblast growth factor-8 | P55075 |
| SEQ ID NO:67 | VILHLTVLL | Transcription factor ELYS | Q8WYPS |
| SEQ ID NO:68 | TIYPKYEVN | Bone morphogenetic protein 1 | P13497 |
| SEQ ID NO:69 | FVIIFLGVVL | Receptor-type protein-tyrosine | P28827 |
| | | phosphatase mu | |
| SEQ ID NO:70 | VTSSIITVPV | Ankyrin 3 (ANK-3) (Ankyrin G) | Q12955 |
| SEQ ID NO:71 | AEDLLRFTV | Sorting nexin 15 | Q9NRS6 |
| SEQ ID NO:72 | LLGNSPVLLL | ABC transporter ABCA6 | Q8N139 |
| SEQ ID NO:73 | NLMEQPIKV | Junction plakoglobin | P14923 |
| SEQ ID NO:74 | ALLRRPTV | G2/mitotic-specific cyclin B2 | O95067 |
| SEQ ID NO:75 | NRIVLPFSF | Jagged 1 | P78504 |
| SEQ ID NO:76 | FSLSPFPSL | Homeobox protein Cux-2 | O14529 |
| SEQ ID NO:77 | ALLREGESL | L-plastin | P13796 |
| SEQ ID NO:78 | ILPVPAFNV | Gamma enolase | P09104 |
| SEQ ID NO:79 | VMIPTLPSI | FRAP-related protein | Q13535 |
| SEQ ID NO:80 | SLEPLLQQL | WNT1 inducible signaling | O95389 |
| | | pathway protein 3 | |
| SEQ ID NO:81 | TXDIDLKAL | discs large homolog 5 | NP_004738.2 |
| SEQ ID NO:82 | MEVRLIFL | Presenilin-like protein 2 | Q8TCT8 |
| SEQ ID NO:83 | YLLPAIVHI | Probable RNA-dependent | P17844 |
| | | helicase p68 | |
| SEQ ID NO:84 | ELNLETPEI | Neuroblast differentiation | Q09666 |
| | | associated protein AHNAK | |
| SEQ ID NO:85 | NIGLAILFV | Junctophilin 1 | Q9HDC5 |
| SEQ ID NO:86 | KGERGLVPTD | Sorting nexin 9 | Q9Y5X1 |
| SEQ ID NO:87 | TLPATVPLLL | Telomerase-associated protein | Q99973 |
| | | TP-1 | |
| SEQ ID NO:88 | ALRKQLVPLL | Desert hedgehog protein | O43323 |
| | | precursor | |
| SEQ ID NO:89 | QAPLLPLL | ephrin-A2 | JE0322 |
| SEQ ID NO:90 | MIEANIRR | Clock protein | Q8TAB0 |
| SEQ ID NO:91 | SLPEVLPIL | Integrin alpha-6 precursor | P23229 |
| | | (VLA-6) | |
| SEQ ID NO:92 | MDKEVDDIL | Serine phosphatase FCP1a | Q9Y6F5 |
| SEQ ID NO:93 | VMAGTIGTIL | glycophorin alpha | 1311302A |
| SEQ ID NO:94 | LARGILLPLI | Sorting nexin 13 | Q9Y5W8 |
| SEQ ID NO:95 | NILPPLLQL | Importin alpha-1 subunit | P52294 |
| SEQ ID NO:96 | EDLEDGEDL | Sorting nexin 2 | O60749 |
| SEQ ID NO:97 | FPLEGPYAAA | Neurogenic locus notch homolog | Q9UM47 |
| | | protein 3 | |
| SEQ ID NO:98 | YLKILNEQ | DNA mismatch repair protein | P20585 |
| | | Msh3 | |
| SEQ ID NO:99 | YIILELME | Proto-oncogene tyrosine-protein | P08922 |
| | | kinase ROS precursor | |
| SEQ ID NO:100 | FFTQTKYTFV | Vascular endothelial-cadherin | P33151 |
| | | precursor (VE-cadherin) | |
| SEQ ID NO:101 | QLDKDSPVSA | Laminin alpha-1 chain precursor | P25391 |
| SEQ ID NO:102 | GKEDTQTLQSL | Nestin | P48681 |
| SEQ ID NO:103 | TKENQEPLR | Nestin | P48681 |
| SEQ ID NO:104 | DKDEVDLLI | NAD-dependent deacetylase | Q96EB6 |
| | | sirtuin 1 | |
| SEQ ID NO:105 | MIRLGLFTV | Frizzled 8 | Q9H461 |
| SEQ ID NO:106 | QILPTLVRL | Importin alpha-2 subunit | P52292 |
| SEQ ID NO:107 | EANSSYFHL | Frizzled 10 | Q9ULW2 |
| SEQ ID NO:108 | QAVRQDPLL | Sorting nexin 17 | Q15036 |
| SEQ ID NO:109 | FLSELTQQL | Macrophage Migration Inhibitory | P14174 |
| | | Factor | |
| SEQ ID NO:110 | YLNDGLWHM | MCT-1 protein | Q9ULC4 |
| SEQ ID NO:111 | QLAQFVHEV | DEAD/H Asp-Glu-Ala-Asp/His | Q96FC9 |
| | | box polypeptide 11 | |
| SEQ ID NO:112 | SLDERPVAV | Bone morphogenetic protein | Q13873 |
| | | receptor type II precursor | |
| SEQ ID NO:113 | FLQTYSHEV | Protocadherin gamma A6 | O60330 |
| | | precursor | |
| SEQ ID NO:114 | FLFQEPRSI | Retinoblastoma-associated | Q9UK61 |
| | | protein (RAP140) | |
| SEQ ID NO:115 | NLFNKYPAL | T-plastin | P13797 |
| SEQ ID NO:116 | DGLSQEQLE | B cell Lymphoma Protein 9 | O00512 |
| | | (legless homolog) | |
| SEQ ID NO:117 | NGGTCEDGIN | Notch protein homolog TAN-1 | P46531 |
| | | precursor | |
| SEQ ID NO:118 | AYVHMVTHF | Bax inhibitor-1 | P55061 |
| SEQ ID NO:119 | TEFGHPAAI | F box/WD repeat protein 8 | Q9P2S5 |
| SEQ ID NO:120 | KMIGPTHGQMTV | Protein-tyrosine kinase JAK1 | P23458 |
| SEQ ID NO:121 | HEIGFGSKV | Dj-1 | Q99497 |
| SEQ ID NO:122 | GKLVSESSDVLPK | Keratin 8/cytokeratins 8 type | P05787 |
| | | II cytoskeletal 8 | |
| SEQ ID NO:123 | HEYLKAFKV | Lactadherin precursor (Milk fat | Q08431 |
| | | globule-EGF factor 8) (Breast | |
| | | epithelial antigen BA46) | |
| SEQ ID NO:124 | HLDEAIHVL | Transcription factor E2-alpha | P15923 |
| SEQ ID NO:125 | SLDTQPKKV | Transcription factor E2-alpha | P15923 |

TABLE 4

SEQ ID NO, Parent Protein Identification and SwissProt
Identification Number for Full-length Immunogen
Sequences 126-242.

| | | SwissProt |
|---|---|---|
| | | Identification |
| SEQ ID NO: | Parent Protein Identification | No. |
| SEQ ID NO:126 | Transcriptional repressor CTCF | P49711 |
| SEQ ID NO:127 | Timeless homolog | Q9UNS1 |
| SEQ ID NO:128 | Galectin-1 | P09382 |
| SEQ ID NO:129 | Bone specific CMF608 | AAQ16156.1 |
| SEQ ID NO:130 | Amelogenin | Q99218 |
| SEQ ID NO:131 | Desmoplakin | P15924 |
| SEQ ID NO:132 | Nuclear receptor co-repressor/ | Q9BZK7 |
| | HDAC3 complex subunit TBLR1 | |
| SEQ ID NO:133 | MEGF6 | O75095 |
| SEQ ID NO:134 | Tyrosine-protein kinase JAK3 | P52333 |
| SEQ ID NO:135 | Trophinin-associated protein (Tastin) | Q12815 |
| SEQ ID NO:136 | Adenomatous polyposis coliprotein | P25054 |
| SEQ ID NO:137 | G2/mitotic-specific cyclin B1 | P14635 |
| SEQ ID NO:138 | Semaphorin 5B precursor | Q9P283 |
| SEQ ID NO:139 | LDL receptor-related protein 6 | O75581 |
| SEQ ID NO:140 | Flightless-I protein homolog | Q13045 |
| SEQ ID NO:141 | Ectoderm-neural cortex-1 protein | O14682 |
| | (ENC-1) | |
| SEQ ID NO:142 | Talin 1 | Q9Y490 |
| SEQ ID NO:143 | Semaphorin 6B precursor (Semaphorin Z) | Q9H3T3 |
| SEQ ID NO:144 | Vimentin | P08670 |
| SEQ ID NO:145 | Galectin-8 | CAA62904.1 |
| SEQ ID NO:146 | Wnt-1 proto-oncogene protein precursor | P04628 |
| SEQ ID NO:147 | Beta-catenin | P35222 |
| SEQ ID NO:148 | programmed cell death 4 isoform 2 | NP_663314.1 |
| SEQ ID NO:149 | Lunatic fringe | AAC51360.1 |
| SEQ ID NO:150 | Keratin, type II cytoskeletal 7 | P08729 |
| SEQ ID NO:151 | Ephrin A1 isoformb precursor | NP_872626.1 |
| SEQ ID NO:152 | ORMDL3 | Q8N138 |
| SEQ ID NO:153 | Leptin receptor precursor (LEP-R) | P48357 |
| SEQ ID NO:154 | Ras-related protein Rab-27A | P51159 |
| SEQ ID NO:155 | FKBP-rapamycin associated protein | P42345 |
| | (FRAP) | |
| SEQ ID NO:156 | Transcription factor Dp-1 | Q14186 |
| SEQ ID NO:157 | Ephrin-B3 precursor | Q15768 |
| SEQ ID NO:158 | Semaphorin 6A precursor | Q9H2E6 |
| SEQ ID NO:159 | Homeobox protein Hox-B3 | P14651 |
| SEQ ID NO:160 | SMAD 2 | Q15796 |
| SEQ ID NO:161 | Sorting nexin 1 | Q13596 |
| SEQ ID NO:162 | Ephrin-B1 precursor | P98172 |
| SEQ ID NO:163 | Eyes absent homolog 1 | Q99502 |
| SEQ ID NO:164 | Mago nashi protein homolog | P50606 |
| SEQ ID NO:165 | hCLOCK | O15516 |
| SEQ ID NO:166 | Protein inhibitor of activated STAT | Q9Y6X2 |
| | protein 3 | |
| SEQ ID NO:167 | Frizzled 7 precursor | O75084 |
| SEQ ID NO:168 | Zinc finger protein 294 | O94822 |
| SEQ ID NO:169 | Leukosialin precursor CD34 | P16150 |
| SEQ ID NO:170 | Ras GTPase-activating protein nGAP | Q9UJF2 |
| SEQ ID NO:171 | Serine-protein kinase ATM | Q13315 |
| SEQ ID NO:172 | Smoothened homolog precursor | Q99835 |
| SEQ ID NO:173 | NF45 protein | Q12905 |
| SEQ ID NO:174 | COUP transcription factor 2 | P24468 |
| SEQ ID NO:175 | Disheveled associated activator of | Q9Y4D1 |
| | morphogenesis 1 | |
| SEQ ID NO:176 | Wnt-7b protein precursor | P56706 |
| SEQ ID NO:177 | Semaphorin 3C precursor | Q99985 |
| SEQ ID NO:178 | E1A-associated protein p300 | Q09472 |
| SEQ ID NO:179 | HIRA protein | P54198 |
| SEQ ID NO:180 | Activin | Q04771 |
| SEQ ID NO:181 | Angiopoietin 1 | Q02763 |
| SEQ ID NO:182 | Transcription factor BTF3 | P20290 |
| SEQ ID NO:183 | Ephrin type-A receptor 7 | Q15375 |
| SEQ ID NO:184 | Bone morphogenetic protein 10 | O95393 |
| SEQ ID NO:185 | Fibroblast growth factor-8 | P55075 |
| SEQ ID NO:186 | Transcription factor ELYS | Q8WYP5 |
| SEQ ID NO:187 | Bone morphogenetic protein 1 | P13497 |
| SEQ ID NO:188 | Receptor-type protein-tyrosine | P28827 |
| | phosphatase mu | |
| SEQ ID NO:189 | Ankyrin 3 (ANK-3) (Ankyrin G) | Q12955 |
| SEQ ID NO:190 | Sorting nexin 15 | Q9NRS6 |
| SEQ ID NO:191 | ABC transporter ABCA6 | Q8N139 |
| SEQ ID NO:192 | Junction plakoglobin | P14923 |
| SEQ ID NO:193 | G2/mitotic-specific cyclin B2 | O95067 |
| SEQ ID NO:194 | Jagged 1 | P78504 |
| SEQ ID NO:195 | Homeobox protein Cux-2 | O14529 |
| SEQ ID NO:196 | L-plastin | P13796 |
| SEQ ID NO:197 | Gamma enolase | P09104 |
| SEQ ID NO:198 | FRAP-related protein | Q13535 |
| SEQ ID NO:199 | WNT1 inducible signaling pathway | O95389 |
| | protein 3 | |
| SEQ ID NO:200 | discs large homolog 5 | NP_004738.2 |
| SEQ ID NO:201 | Presenilin-like protein 2 | Q8TCT8 |
| SEQ ID NO:202 | Probable RNA-dependent helicase p68 | P17844 |
| SEQ ID NO:203 | Neuroblast differentiation associated | Q09666 |
| | protein AHNAK | |
| SEQ ID NO:204 | Junctophilin 1 | Q9HDC5 |
| SEQ ID NO:205 | Sorting nexin 9 | Q9Y5X1 |
| SEQ ID NO:206 | Telomerase-associated protein TP-1 | Q99973 |
| SEQ ID NO:207 | Desert hedgehog protein precursor | O43323 |
| SEQ ID NO:208 | Ephrin-A2 | JE0322 |
| SEQ ID NO:209 | Clock protein | Q8TAB0 |
| SEQ ID NO:210 | Integrin alpha-6 precursor (VLA-6) | P23229 |
| SEQ ID NO:211 | Serine phosphatase FCP1a | Q9Y6F5 |
| SEQ ID NO:212 | Glycophorin alpha | 1311302A |
| SEQ ID NO:213 | Sorting nexin 13 | Q9Y5W8 |
| SEQ ID NO:214 | Importin alpha-1 subunit | P52294 |
| SEQ ID NO:215 | Sorting nexin 2 | O60749 |
| SEQ ID NO:216 | Neurogenic locus notch homolog | Q9UM47 |
| | protein 3 | |
| SEQ ID NO:217 | DNA mismatch repair protein Msh3 | P20585 |
| SEQ ID NO:218 | Proto-oncogene tyrosine-protein kinase | P08922 |
| | ROS precursor | |
| SEQ ID NO:219 | Vascular endothelial-cadherin | P33151 |
| | precursor (VE-cadherin) | |
| SEQ ID NO:220 | Laminin alpha-1 chain precursor | P25391 |
| SEQ ID NO:221 | Nestin | P48681 |
| SEQ ID NO:222 | NAD-dependent deacetylase sirtuin 1 | Q96EB6 |
| SEQ ID NO:223 | Frizzled 8 | Q9H461 |
| SEQ ID NO:224 | Importin alpha-2 subunit | P52292 |
| SEQ ID NO:225 | Frizzled 10 | Q9ULW2 |
| SEQ ID NO:226 | Sorting nexin 17 | Q15036 |
| SEQ ID NO:227 | Macrophage Migration Inhibitory Factor | P14174 |
| SEQ ID NO:228 | MCT-1 protein | Q9ULC4 |
| SEQ ID NO:229 | DEAD/H Asp-Glu-Ala-Asp/His box 11 | Q96FC9 |
| SEQ ID NO:230 | Bone morphogenetic protein receptor | Q13873 |
| | type H precursor | |
| SEQ ID NO:231 | Protocadherin gamma A6 precursor | O60330 |
| | (PCDH-gamma-A6) | |
| SEQ ID NO:232 | Retinoblastoma-associated protein | Q9UK61 |
| | (RAP 140) | |
| SEQ ID NO:233 | T-plastin | P13797 |
| SEQ ID NO:234 | B cell Lymphoma Protein 9 (legless | O00512 |
| | homolog) | |
| SEQ ID NO:235 | Notch protein homolog TAN-1 precursor | P46531 |
| SEQ ID NO:236 | Bax inhibitor-1 | P55061 |
| SEQ ID NO:237 | F box/WD repeat protein 8 | Q9P2S5 |
| SEQ ID NO:238 | Protein-tyrosine kinase JAK1 | P23458 |
| SEQ ID NO:239 | Dj-1 | Q99497 |
| SEQ ID NO:240 | Keratin 8/cytokeratins 8 type II | P05787 |
| | cytoskeletal 8 | |
| SEQ ID NO:241 | Lactadherin precursor (Milk fat | Q08431 |
| | globule-EGF factor 8) (Breast | |
| | epithelial antigen BA46) | |
| SEQ ID NO:242 | Transcription factor E2-alpha | P15923 |

We claim:

1. An isolated oligopeptide or peptide comprising at least one peptide having an amino acid sequence selected from the group consisting of SEQ ID NO: 1-125, said oligopeptide or peptide consisting of 8 to about 50 amino acid residues, wherein said oligopeptide or peptide binds to class I MHC molecules or can be processed to bind to class I MHC molecules.

2. The oligopeptide of claim 1 wherein said oligopeptide comprises at least two epitopic peptides.

3. The oligopeptide of claim 1 wherein said oligopeptide comprises at least three epitopic peptides.

4. The oligopeptide of claim 1 wherein said oligopeptide comprises at least four epitopic peptides.

5. The oligopeptide or peptide of claim 1 wherein said oligopeptide or peptide differs from SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 wherein said difference is no more than one amino acid unit.

6. The oligopeptide or peptide of claim 5 wherein said one amino acid difference is the result of a conservative amino acid substitution.

7. The oligopeptide or peptide of claim 5 wherein said one amino acid difference is the substitution of one hydrophobic amino acid with another hydrophobic amino acid.

8. The oligopeptide or peptide of claim 5 wherein said amino acid difference is the addition or deletion of one amino acid to or from said epitopic peptide.

9. A polynucleotide comprising a polynucleotide selected from the group consisting of:
(a) a polynucleotide that encodes an oligopeptide or peptide of claim 1, and
(b) the full complement of (a).

10. The polynucleotide of claim 9 wherein the polynucleotide of (a) is DNA.

11. The polynucleotide of claim 9 wherein the polynucleotide of (a) is RNA.

12. A method for treating a subject with cancer, said cancer characterized by the presence of cancer stem cells, said cancer stem cells expressing any class I MHC molecule, comprising administering to said subject a composition that binds to class I MHC molecules or can be processed to bind to class I MHC molecules comprising:
at least one polypeptide comprising an epitopic peptide comprising an amino acid sequence selected from the group consisting of SEQ ID NO: 1 to 242 in an amount sufficient to induce a CTL response to said tumor cells; or
at least one polypeptide comprising an epitopic peptide having at least one amino acid difference from an amino acid sequence selected from the group consisting of SEQ ID NO: 1 to 242 in an amount sufficient to induce a CTL response to said tumor cells.

13. A method for treating a subject with cancer, said cancer characterized by the presence of cancer stem cells, said cancer stem cells expressing any class I MHC molecule, said method comprising administering to said subject a composition that binds to class I MHC molecules or can be processed to bind to class I MHC molecules comprising:
a polynucleotide comprising a nucleic acid sequence encoding at least one polypeptide comprising an amino acid sequence selected from the group consisting of SEQ ID NO: 1 to 242 in an amount sufficient to induce a CTL response to said tumor cells; or
at least one polypeptide comprising an epitopic peptide comprising one amino acid difference from an amino acid sequence selected from the group consisting of SEQ ID NO: 1 to 242 in an amount sufficient to induce a CTL response to said tumor cells.

14. A method for treating a subject with cancer, said cancer characterized by the presence of cancer stem cells, said method comprising:
stimulating the production of antibodies for use in passive immunotherapy, wherein said antibodies react with at least one polypeptide comprising an amino acid sequence selected from the group consisting of SEQ ID NO: 126 to 242; or
at least one polypeptide comprising one amino acid difference from an amino acid sequence selected from the group consisting of SEQ ID NO: 126 to 242.

15. The method of claim 14, wherein said antibodies are recombinant antibodies.

16. A method for diagnosing the presence of cancer stem cells in a subject comprising obtaining a tissue sample from said subject; and detecting at least one polypeptide comprising an amino acid sequence selected from the group consisting of SEQ ID NO: 126 to 242 in said sample.
